(19) United States
(12) Patent Application Publication
(10) Pub. No.: US 2013/0324617 A1
(43) Pub. Date: Dec. 5, 2013

(54) CROWN-LIKE STRUCTURES AS A BIOMARKER FOR CANCER RISK AND CANCER PROGNOSIS

(71) Applicants: Andrew J. Dannenberg, New York, NY (US); Kotha Subbaramaiah, New Hyde Park, NY (US); Clifford Hudis, New York, NY (US)

(72) Inventors: Andrew J. Dannenberg, New York, NY (US); Kotha Subbaramaiah, New Hyde Park, NY (US); Clifford Hudis, New York, NY (US)

(73) Assignees: MEMORIAL SLOAN-KETTERING CANCER CENTER, New York, NY (US); CORNELL UNIVERSITY, Ithaca, NY (US)

(21) Appl. No.: 13/984,939

(22) PCT Filed: Feb. 11, 2012
(86) PCT No.: PCT/US12/24777

§ 371 Date: Aug. 12, 2013

Related U.S. Application Data
(60) Provisional application No. 61/441,990, filed on Feb. 11, 2011.
(60) Provisional application No. 61/470,071, filed on Mar. 31, 2011.

(51) Int. Cl. G01N 33/50

(52) U.S. Cl. CPC G01N 33/5091
USPC 514/733; 435/7.23; 435/25

(57) ABSTRACT

Chronic inflammation increases the risk of several epithelial malignancies. The present invention provides methods for determining cancer risk in a patient which comprises detecting the presence of crown-like structures (CLS), methods for treating cancer associated with CLS presence, methods for determining cancer risk in a patient by quantifying the number of CLS in a sample of adipose tissue of the patient, methods of determining prognosis of a patient with breast cancer or other cancers by detecting CLS, and screening methods for anti-cancer agents or agents that inhibit or reduce CLS formation or consequences thereof.

### CROSS-REFERENCE TO RELATED APPLICATIONS

This application claims priority under 35 U.S.C. §119(e) to provisional applications U.S. Ser. No. 61/441,990, filed Feb. 11, 2011, and U.S. Ser. No. 61/470,071, filed Mar. 31, 2011, each of which is incorporated herein in its entirety.

### FIELD OF THE INVENTION

Chronic inflammation increases the risk of several epithelial malignancies. Obesity is a risk factor for the development of hormone receptor (HR)-positive breast cancer in postmenopausal women and has been associated with an increased risk of recurrence and reduced survival. In humans, obesity causes subclinical inflammation in visceral and subcutaneous adipose tissue, characterized by necrotic adipocytes surrounded by macrophages forming crown-like structures (CLS). The discovery of increased numbers of CLS in the mammary glands of obese mice and breast of women was associated with increased levels of pro-inflammatory mediators, which were paralleled by elevated levels of aromatase. Further investigations have shown a relationship between CLS and other malignancies.

### BACKGROUND OF THE DISCLOSURE

More than 40,000 women in the U.S. die each year of metastatic breast cancer. Approximately two-thirds of breast cancer patients have tumors that express estrogen receptors. Obese postmenopausal women are at significantly increased risk of developing HR-positive breast cancer (Calle 2004). Estrogens are synthesized from androgens in a reaction catalyzed by cytochrome P450 aromatase (aromatase), encoded by the CYP19 gene (Santen 2009). After menopause, peripheral aromatization of androgen precursors in adipose tissue is largely responsible for estrogen biosynthesis (Lorincz 2006). The increased risk of HR-positive breast cancer in obese postmenopausal women has been attributed, in part, to elevated levels of circulating estradiol related to both increased adipose tissue and elevated aromatase expression in subcutaneous adipose tissue (Cleary 2009, Key 2003, van Kruijsdijk 2009, Wake 2007). Whether obesity alone leads to changes in aromatase expression in breast tissue is uncertain.

Because of the significance of estrogen biosynthesis in the pathogenesis of HR-positive breast cancer, efforts have been made to elucidate the mechanisms that regulate the transcription of CYP19 (Bulun 2005). Multiple lines of evidence suggest a key role for pro-inflammatory mediators including TNF-α, IL-1β and cyclooxygenase (Cox)-derived prostaglandin E₂ (PGE₂) in stimulating CYP19 transcription resulting in increased aromatase activity (Brodie 2001, Irahara 2006, Karuppu 2002, Subbaramaiah 2006, Zhao 1996, Zhao 1997, Purohit 2002, Hardy 2008, Salama 2009). Obesity causes inflammation and increased levels of pro-inflammatory mediators in adipose tissue (van Kruijsdijk 2009, Cancello 2005, Cinti 2005, Olefsky 2010). In humans, a correlation has been observed between increased body mass index and elevated levels of aromatase in subcutaneous fat (Wake 2007). This constellation of findings suggested the possibility that obesity-related inflammation is causally linked to increased aromatase expression. Although studies have shown that obesity causes inflammation in both visceral and subcutaneous fat (Olefsky 2010), it is unknown whether similar changes occur in the mammary gland.

In addition to its impact on breast cancer risk, obesity has been recognized as a poor prognostic factor among breast cancer survivors (Calle 2003, Whiteman 2005, Majed 2008, Petrelli 2002, Ewertz 2011, Roxe 2009, Protani 2010). Obesity-related effects on hormones, adipokines and pro-inflammatory mediators have been suggested to contribute to the worse prognosis of obese patients (Sinicrope 2011, McTiernan 2010, Duggan 2011, Goodwin 2002).

Obesity causes subclinical inflammation in adipose tissue (van Kruijsdijk 2009, Cancello 2005, Cinti 2005, Olefsky 2010). In both mouse models of obesity and obese humans, macrophages infiltrate visceral and subcutaneous adipose tissue and form characteristic CLS around necrotic adipocytes (Cinti 2005, Olefsky 2010, Murano 2008, Weisberg 2003). These macrophages produce pro-inflammatory mediators including TNF-α, IL-1β, IL-6 and PGE₂, which may contribute to insulin resistance (Olefsky 2010, Suganami 2010, Xu 2003, Kern 2001). In obese women, increased levels of pro-inflammatory mediators are commonly found in the circulation and may contribute to breast cancer progression and mortality (Bachelot 2003, Dandona 1998, Vosarova 2001).

The present invention shows, in both dietary and genetic models of obesity, that CLS occur in the adipose tissue of the mouse mammary gland (termed CLS-B) in addition to visceral fat and it has been discovered that CLS-B can serve as a biomarker for increased breast cancer risk or poor prognosis. Moreover, increased expression of aromatase in the mammary adipose tissue may explain why the recommended doses of aromatase inhibitor are less effective in the treatment of HR-positive breast cancer in obese vs. lean women (Sestak 2010). The discovery that obesity leads to inflammation which in turn increases aromatase activity provides the basis for developing mechanism-based strategies to reduce the risk of HR-positive breast cancer in this growing segment of the population. Additionally, CLS have been observed in periprostatic adipose tissue in a mouse genetic obesity model (termed CLS-P) and neck adipose tissue in diet-induced obese mice (termed CLS-N) as well as in neck adipose tissue in patients with oral squamous cell carcinoma, where presence of CLS-N also correlate with obesity and risk of lymph node metastasis.

### SUMMARY OF THE INVENTION

The discovery of the obesity-inflammation-aromatase axis in mammary gland and its association with CLS in mice provides insight into potential mechanisms underlying the increased cancer risk. Accordingly, the present invention is directed to a method for determining cancer risk or prognosis in a patient by detecting the presence of CLS in adipose tissue in or adjacent to epithelial-derived tissue or organs such as the breast, prostate, kidney, pancreas, liver, colon and the like as well as in adipose tissue associated with a lymph node metastasis arising from an epithelial-derived cancer. The presence of CLS indicates poor prognosis or an increased cancer risk. CLS are detected in a tissue sample that has been fixed, sectioned and stained either histochemically or with a macrophage-specific marker so that the CLS surrounding adipocytes in the section can be identified. The presence and density of the CLS can be quantitated.

The samples for use in the method of the invention are generally obtained during cancer surgery but can also be from tissue biopsies. These samples will contain adipose tissue as well other tissue types or cells associated with the excised tissue, tumor or organ. In some embodiments, the cancer risk or prognosis is for breast cancer. In other embodiments, the cancer risk or prognosis is for a head and neck carcinoma. In other embodiments, the cancer risk or prognosis is for a cancer of epithelial origin, including but not limited to, prostate cancer, colorectal cancer, kidney cancer, pancreatic cancer or liver cancer.

Further, the method of detecting CLS can be combined with determining a patient's body mass index (BMI), levels of pro-inflammatory mediators (such as, e.g., TNF-α. IL-1β or COX-2), aromatase expression levels or aromatase activity, NF-κB activity or binding, or any combination thereof. The presence of CLS either alone or together with any of higher patient BMI, elevated levels of pro-inflammatory mediators relative to a normal tissue, increased aromatase expression relative to normal tissue or elevated levels of aromatase activity relative to normal tissue indicates increased cancer risk or poor prognosis.

A still further aspect of the instant invention relates to a method for determining cancer prognosis or cancer risk in a patient, which comprises detecting and quantifying the number of CLS in a sample of adipose tissue of the patient, wherein individuals with a CLS index of 0.2 to 1.0 have a worse prognosis or are at greater risk for cancer than individuals with a CLS index of less than 0.2. This method can also be combined with determining a patient's body mass index (BMI), levels of pro-inflammatory mediators (such as, e.g., TNF-α. IL-1β or COX-2), aromatase expression levels or aromatase activity or any combination thereof. Similarly, presence of any of the foregoing with CLS also indicates an increased cancer risk or a poor prognosis.

Another aspect of the invention provides a method for treating cancer in a patient by determining whether CLS are present in adipose tissue of a patient by detecting the presence of CLS in that adipose tissue. The sampled adipose tissue is from a tissue sample found in or around an epithelial-derived tissue or organ or from adipose tissue associated with a lymph node metastasis arising from an epithelial-derived cancer. When CLS are detected, the patient is administered a therapeutically-effective dose of a therapeutic agent that reduces the number or function of CLS or the biological consequences of CLS, or any combination thereof. Such therapeutic agents include, but are not limited to, a Toll-like receptor antagonist, an NF-κB inhibitor, a CDK5 inhibitor, a TNF-α inhibitor, a COX-2 inhibitor, an IL-1 inhibitor, an aromatase inhibitor, a PI3K inhibitor, a Rho-kinase inhibitor, an Akt inhibitor, an mTOR inhibitor, an IGF-1R antagonist, an anti-leptin compound, an AMPK activator, a statin, and resveratrol or other polyphenols. CLS are detected and quantitated in accordance with the invention. The cancers that can be treated include, but are not limited to, breast cancer, head and neck carcinoma, particularly, squamous cell carcinoma, prostate cancer, colorectal cancer, kidney cancer, pancreatic cancer and liver cancer.

Yet a further aspect of the invention provides a method for treating CLS-related cancer by administering a therapeutic agent that alters the number or function of CLS or its biological consequences to a patient for a time and in an amount to improve prognosis, to reduce risk of cancer recurrence, to regress tumor or to reduce metastasis. In some embodiments, the therapeutic agent is selected from the group consisting of a Toll-like receptor antagonist, an NF-κB inhibitor, a CDK5 inhibitor, a TNF-α inhibitor, a COX-2 inhibitor, an IL-1 inhibitor, an aromatase inhibitor, a PI3K inhibitor, a Rho-kinase inhibitor, an Akt inhibitor, an mTOR inhibitor, an IGF-1R antagonist, an anti-leptin compound, an AMPK activator, a statin, resveratrol or other polyphenols. A useful aromatase inhibitor is exemestane.

In another embodiment, the invention is directed to a method of determining prognosis of a patient with breast cancer by detecting CLS in breast adipose tissue, wherein presence of CLS indicates a worse prognosis. CLS can be detected by obtaining a tissue sample, preparing one or more tissue sections from the sample, staining those sections histochemically or with a macrophage-specific marker, preferably a marker for CD68, and identifying CLS therein. If needed, the CLS can be quantitated by calculating a CLS-index for the patient. Those individuals with high numbers of CLS or higher CLS-index tend to have a worse prognosis or are at higher risk for having or developing hormone receptor-positive breast cancer than individuals with low numbers of CLS, no CLS or a lower CLS-index. Those individuals with breast cancer and higher numbers of CLS or higher CLS-index also have a worse prognosis. The association of risk and hormone receptor-positive breast cancer is strong in postmenopausal women.

The invention also provides a method of determining prognosis of a patient with a head and neck carcinoma by detecting CLS in adipose tissue found with lymph node metastasis associated with said carcinoma, wherein presence of CLS indicates a worse prognosis. In some embodiments, the head and neck carcinoma is a squamous cell carcinoma, including oral squamous cell carcinoma.

Still yet the invention is directed to an in vitro method to screen a compound for anti-cancer activity by (a) culturing macrophages with the test compound and a saturated fatty acid in an amount and for a time sufficient to produce conditioned medium comprising pro-inflammatory mediators; (b) harvesting the conditioned medium; (c) culturing cells selected from the group consisting of preadipocytes, adipocytes, fibroblasts, epithelial cells and tumor cells in the presence of the conditioned medium for a time to induce a cancer-related response from the cells; and (d) detecting that response, wherein inhibition or reduction of the response, relative to similarly treated control cultures without the compound, indicates the compound has anti-cancer activity. Examples of cancer-related responses for the assays of the invention include, but are not limited to, inducing aromatase activity, increasing cell proliferation, increasing in cell motility, increasing invasiveness, increasing NFκB activity, or decreasing apoptosis. For example, when the method is used with preadipocytes, it is convenient to assay induction of aromatase activity, so that a compound with anti-cancer activity inhibits or reduces aromatase activity, which is detected by the method of the invention. Similarly, when tumor cells are used, it is convenient to assay for increases in cell proliferation, increases in cell motility, increases in invasiveness, increases in NFκB activity, or decreases in apoptosis.

In an alternative embodiment of the foregoing assay, instead of culturing the macrophages with the test compound in step (a), the test compound cultured with the cells in step (c). Otherwise the method is conducted in the same manner.

In both of these in vitro screening methods, the saturated fatty is selected from the group consisting of lauric acid, myristic acid, palmitic acid and stearic acid. Also in both techniques, aromatase activity means either the level of aromatase expression in the cells or the enzymatic activity of aromatase by the cells.

Yet further embodiments provide a method for determining a compound's activity against CLS by screening a compound in a dietary or genetic model of obesity in mice and determining whether the compound inhibits or reduces formation of CLS in adipose tissue of the mice or a functional consequence of CLS in the mice, especially in breast adipose tissue. The method is useful for finding compounds that have anti-cancer activity, that are anti-diabetogenic or that have anti-obesity activity. Such compounds that inhibit or reduce formation of CLS are considered potential cancer therapeutics.

In a more particular embodiment, the invention is directed to a screening method to identify a compound with a medical benefit by treating lean mice and obese mice with the test compound in an amount and for a time sufficient to assess the effects of said compound and determining whether the compound preferentially inhibits or reduces formation of CLS-B or functional consequences of CLS-B in the obese mice, wherein a compound which inhibits or reduces formation of CLS-B or functional consequences of CLS-B provides a medical benefit. In one embodiment, to increase the differential effects of the test compound, the lean mice are ovary-intact mice fed a low fat diet and the obese mice are ovariectomized mice fed a high fat diet. The medical benefits include, but are not limited to, anti-cancer activity, anti-diabetogenic activity or anti-obesity activity.

Both this and the foregoing in vivo screening model can be used in conjunction with a tumor formation model, wherein the mice are injected with tumor cells before (e.g., to allow tumor establishment and monitor regression) or simultaneously with administering of the test compounds (e.g., to assess inhibition of tumor formation).

The test compounds for use in the in vitro and in vivo screening methods can be a small molecule, a protein, a peptide, an oligonucleotide, an siRNA and the like. Any of the test compounds may be stabilized or otherwise treated to enhance stability or increase uptake in the cells or by the animals.

### BRIEF DESCRIPTION OF THE DRAWINGS

FIG. 1 shows the results of diet-induced obesity on inflammation in the mammary gland and visceral fat for ovary intact and ovariectomized (OVX) female mice fed a low fat (LF) or high fat (HF) diet for 10 weeks. Panel A provides a bar graph with the average weight of mice shown as mean±SD. Panel B is a photograph of a hematoxylin and eosin (H+E) stained slide showing the presence of an inflammatory focus containing macrophages surrounding a necrotic adipocyte (arrow, 200×). This is a CLS-B. Panels C and D are box-plots of the number of inflammatory foci in mammary glands and visceral fat of mice in the different treatment groups. Abbreviations: LF, ovary-intact mice fed an LF diet; LF+OVX, ovariectomized mice fed an LF diet; HF, ovary-intact mice fed an HF diet; HF+OVX, ovariectomized mice fed an HF diet.

FIG. 2 show the effects of diet-induced obesity on levels of pro-inflammatory mediators in the mammary gland and visceral fat. Box-plots of TNF-α, IL-1β and Cox-2 mRNA expression in mammary glands (A-C) and visceral fat (D-F) are shown. Significant differences were observed across the four experimental groups for each pro-inflammatory mediator (P<0.005). Abbreviations as in FIG. 1.

FIG. 3 show the effects of diet-induced obesity on aromatase expression and activity in the mammary gland and visceral fat. Box-plots of relative aromatase mRNA levels and activity in mammary glands (A, B) and visceral fat (C, D) of mice are shown. Significant differences were observed across the four experimental groups for aromatase expression and activity (P<0.001). Aromatase activity expressed as femtomoles/μg protein/hour. Abbreviations as in FIG. 1.

FIG. 4 graphically illustrates that treating macrophages with saturated fatty acids causes dose-dependent induction of the pro-inflammatory mediator TNF-α. Similar results were obtained for the pro-inflammatory mediators IL-1β and Cox-2. THP-1 cells were treated with the indicated concentration of saturated fatty acid (LA, lauric acid; MA, myristic acid; PA, palmitic acid; SA, stearic acid) for 24 hours and real-time PCR was used to quantify mRNAs. Columns, means (n=6); bars, SD. *P<0.05.

FIG. 5 is a schematic diagram of the human study consort.

FIG. 6 provides a photograph of a CLS of the breast (CLS-B) occurring in human breast tissue. The cells are stained with H+E and show an inflammatory focus containing macrophages that surround a necrotic adipocyte (200×). Arrow indicates CLS-B.

FIG. 7 is a photograph illustrating immunohistochemical stain with CD68 of the same lesion shown in FIG. 6 but taken approximately 30 microns from the section shown in that panel. This staining confirms that the cells constituting CLS-B are macrophages (200×). Arrow indicates CLS-B.

FIG. 8 shows a bar graph indicating that overweight and obesity are associated with the presence of CLS-B. Normal, BMI 18.5-24.9 (N=12); overweight, BMI 25-29.9 (N=10); obese, BMI≧30 (N=8).

FIG. 9 is plot showing that increasing BMI is associated with increased CLS-B intensity (measured as the proportion of blocks positive for CLS-B) using logistic regression. N=30.

FIG. 10 shows H+E staining on a section of prostate tissue from lean mice (control) at magnifications of 40×, 100× and 200×.

FIG. 11 shows H+E staining on a section of prostate tissue from obese male ob/ob mice at magnifications of 40×, 100× and 200×. The arrows (at 100× and 200×) indicate the presence of crown-like structures in the periprostatic adipose tissue (CLS-P).

FIG. 12 is a bar graph showing the correlation of BMI with percent of head and neck squamous cell carcinoma cases with CLS-N. Note that percentage of cases with CLS-N increases with BMI.

### DETAILED DESCRIPTION OF THE INVENTION

### Overview and Definitions

It was discovered that the presence of CLS was associated with increased levels of pro-inflammatory mediators, which were paralleled by elevated levels of aromatase expression and activity in both the mammary gland and visceral fat, leading to the conclusion that the obesity-inflammation-aromatase axis may contribute to the increased risk of hormone receptor-positive breast cancer in postmenopausal women, and the generally worse prognosis of obese breast cancer patients. Hence, the presence of CLS may be a biomarker of increased breast cancer risk or poor prognosis. Although CLS occur in both visceral and subcutaneous fat in overweight and obese women, nothing is known about the existence of CLS of the breast (CLS-B) in humans. This invention establishes that CLS-B occurs in humans and that their presence is associated with obesity. Moreover, the severity of breast inflammation (CLS-B intensity) varies according to body mass index (BMI), and given the link between chronic inflammation and cancer risk in other tissues (Murano 2008), obesity-mediated inflammation in the breast, as indicated by the presence of CLS-B, appears to contribute to both increased risk of breast cancer and worse prognosis in patients with breast cancer.

The invention further establishes that obesity is associated with increased levels of both pro-inflammatory mediators and aromatase in the mammary gland and visceral fat. Increased levels of aromatase mRNA and activity paralleled the elevated levels of TNF-α, IL-1β and Cox-2 in the mammary gland and visceral fat of obese mice. Further, activation of macrophages, a component of the inflammatory cell infiltrate in adipose and mammary tissues of obese mice, led to increased production of pro-inflammatory mediators which contribute, in turn, to the induction of aromatase. Collectively, these results, without being bound to a mechanism, provide potential insights into why obese postmenopausal women are at increased risk of developing HR-positive breast cancer. Moreover, overexpression of aromatase in the adipose tissue of obese mice offers an explanation for why the recommended doses of aromatase inhibitor are less effective in the treatment of HR-positive breast cancer in obese vs. lean women (Sestak 2010) and improved methods for treating such women.

Accordingly, this disclosure presents methods for determining cancer prognosis, cancer risk, and resistance to cancer therapeutics by identification of the presence and amount of CLS in adipose tissue, typically in adipose tissue found in or around an epithelial-derived tissue or organ or in adipose tissue associated with a lymph node metastasis arising from an epithelial-derived cancer. The inventors have discovered that CLS are indicative of increased cancer risk, particularly for cancers of epithelial-derived tissues that contain and/or are proximate to adipose tissue or cells in a subject. CLS are also herein identified as a biomarker for determining resistance to cancer therapeutics such as aromatase inhibitors, and as a biomarker for use in screening methods to identify candidate compounds for cancer treatment or prevention.

“CLS” or “crown-like structures” in accordance with the invention are inflammatory foci or lesions comprised of macrophages surrounding an adipocyte, typically a necrotic or dead adipocyte in adipose tissue. As used herein, CLS found in and around white adipose tissue associated with breast tissue, mammary glands and the like are called CLS-B. CLS found in periprostatic tissue are termed CLS-P. CLS have also been found in white adipose tissue associated with lymph node metastasis in the necks of patients that have oral squamous cell carcinoma. Hence, CLS found in adipose tissue associated with lymph node metastasis arising from an epithelial-derived cancer are termed CLS-N. The number of macrophages found in CLS is not typically fixed but does appear to vary between about 6-10 and about 20-30.

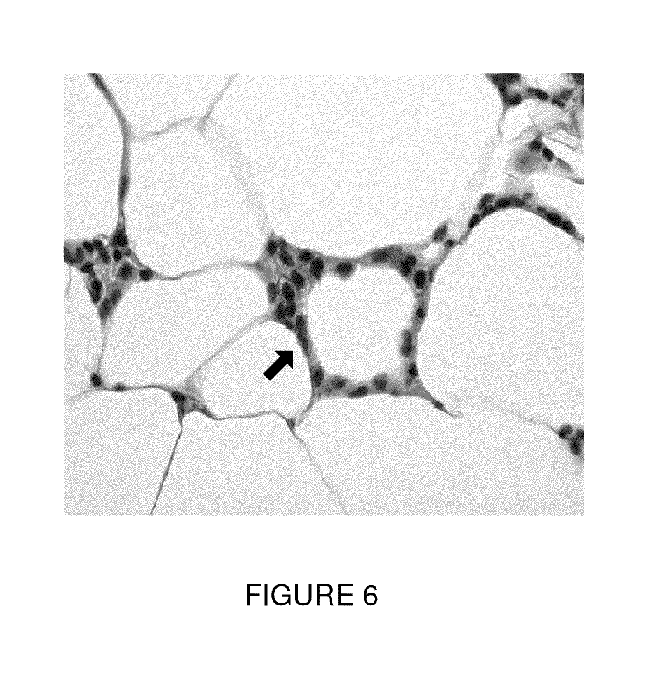

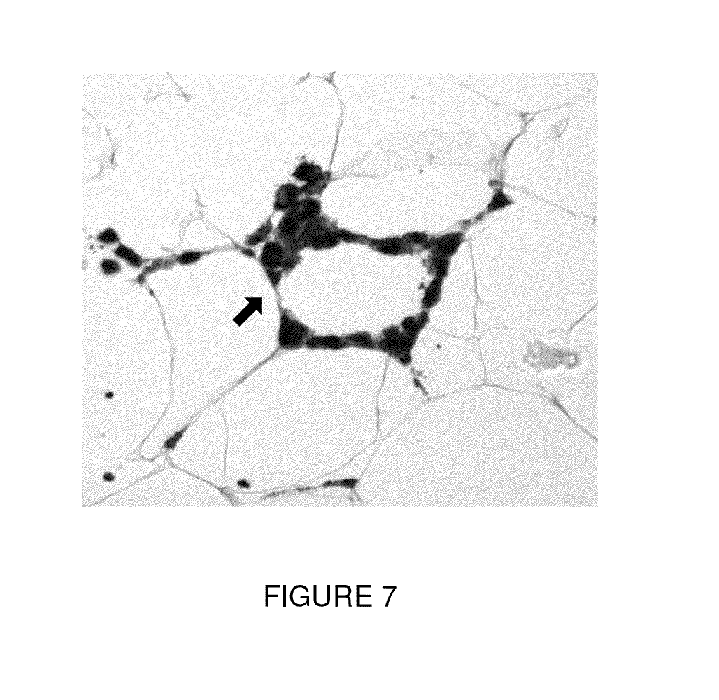

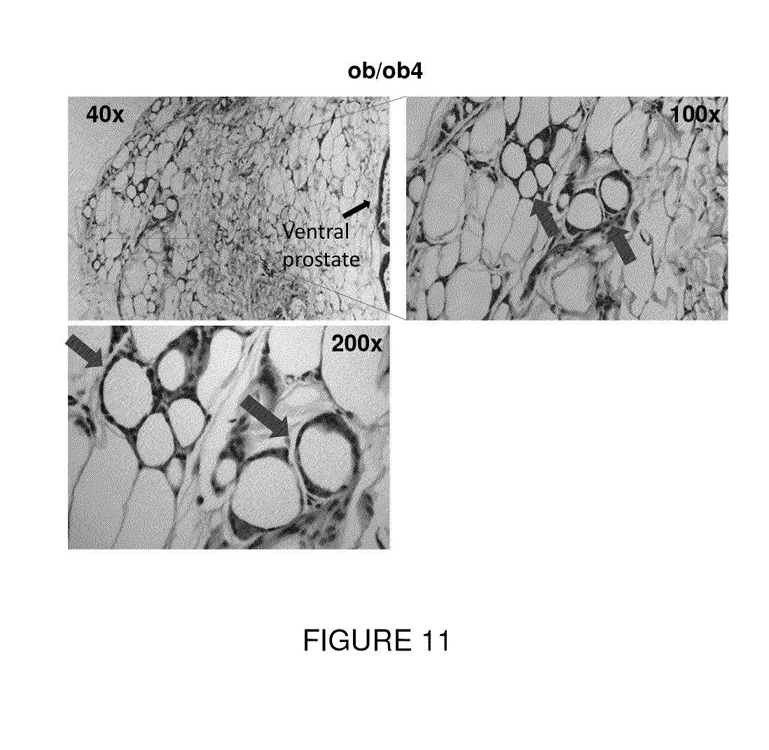

Examination of tissue sections prepared from patient samples by standard histological techniques, or by staining tissue sections using macrophage-specific markers, reveals CLS as a lesion with a “crown-like” morphology surrounding adipose cells, independent of the adipose tissue in which it is shown. By way of examples representative of all CLS, histologically-stained CLS-B are shown in FIG. 6, immunohistochemically-stained CLS-B in FIG. 7 and CLS-P in FIG. 11. Adipose cells displaying CLS are typically necrotic or dead.

CLS can be identified by histological preparation and examination of adipose cells in visceral fat or subcutaneous fat. Methods of histological staining, including tissue fixation, sectioning and staining are well known in the art. One staining method is the use of hematoxylin and eosin (H+E). Immunohistochemical staining techniques can also be used to identify CLS in tissue sections and tend to produce clearer results than H+E (perhaps due to increased staining intensity). Use of immunological markers specific for macrophages can be used in these techniques. For example, antibodies against CD68, a human macrophage marker, provide good detection of CLS in tissue sections. Other macrophage-specific markers can be used, including the markers CCL2 (MCP-1) and CCL3 (macrophage inflammatory protein [MIP]-1α). Immunohistochemical markers are typically antibodies specific for the particular marker. However, other marker types can be used. Macrophage markers may also be specific for the species in which CLS are being detected.

CLS is a biomarker that correlates with increased inflammation adjacent to a tumor or in a tumor-free fat depot. Inflammation around an area of neoplastic cells, particularly for tumors of epithelial origin surrounded by adipocytes (as found in fat deposits on or around a tumor, including lymph node metastases, particularly tumors associated with organs), indicates a cancer subtype that is exacerbated by the presence of pro-inflammatory mediators and increased presence of hormones and hormone precursors. Once this subtype of cancer is identified, a differential diagnosis and treatment regime can be applied to the patient, relative to diagnosis and treatment of a patient who does not present with CLS.

The presence of CLS is associated with activation of NF-κB and increased levels of pro-inflammatory mediators (TNF-α, IL-1β, Cox-2), which are paralleled by elevated local levels of aromatase. The discovery of the obesity-inflammation-axis and its association with CLS in mice provides insight into potential mechanisms underlying the increased risk of breast cancer in obese postmenopausal women.

In one embodiment, identification of CLS begins with identifying the presence or absence of any CLS structures in a patient tissue sample containing and/or adjacent to tumor tissue or from the fat adjacent to tumor tissue. CLS are identified by infiltration of macrophages surrounding adipose cells in sections of the tissue. If any CLS are found in the patient sample, the patient is identified as CLS-positive. Absence of CLS classifies the patient as CLS negative.

CLS-positive patients can be further categorized by CLS index. “CLS index” is a measurement of the fraction of preparations, from multiple samples or tissue sections from a single patient, that show positive macrophage staining. The index scores the number of histological preparations in which a CLS is identified, relative to the total number of preparations examined for a patient. For example, if five patient samples are collected, or one patient sample is divided to prepare five histological specimens, and CLS are identified in three of the five preparations examined, the CLS index for that patient is 3/5 or 0.6. If CLS are identified in all five preparations, the CLS index is 1.0. If no CLS are identified in any preparations (CLS-negative), the CLS index for that patient is 0.0. A CLS index of 0.2 to 1.0 indicates poor prognosis and increased risk of hormone receptor-positive cancer.

Accordingly, the present invention is directed to a method for determining cancer risk or prognosis in a patient by detecting the presence of CLS associated with fat or tissue surrounding, near or adjacent to a suspected or confirmed tumor in the patient. The presence of CLS indicates poor prognosis or an increased cancer risk. CLS are detected in a tissue sample that has been fixed, sectioned and stained either histochemically or with a macrophage-specific marker so that the CLS in the section can be identified. The presence and density of the CLS can be quantitated as described herein.

“Epithelial-derived tissues” are tissues and cells covering internal and external body surfaces (cutaneous, mucous and serous), including the glands and other structures derived therefrom. Exemplary epithelial tissue includes: esophageal epithelium; glandular epithelium, which refers to epithelium composed of secreting cells; and squamous epithelium, which refers to epithelium composed of flattened plate-like cells. The term epithelium can also refer to transitional epithelium, such as that characteristically found lining hollow organs. In particular, the epithelial-derived tissues that are the focus of the invention are those that contain, or are found adjacent to or in close proximity with, adipose cells and tissues. Epithelial-derived tissues and organs that are the subject of the invention include, for example, breast, prostate, oral cavity (e.g., tongue), pancreas, liver, colon and others.

Adipose tissue, unless qualified in context, includes both visceral fat and subcutaneous fat. It may be composed of white adipose tissue or brown adipose tissue. Adipose tissue is found in many places throughout the body, and includes adipose tissue in the breast or other organs, adipose tissue that may be disposed on or around organs, such as periprostatic tissue or peripancreatic tissue, and adipose tissue found near or associated with the lymphoid system and particularly, for purposes of the present invention, adipose tissue associated with lymph nodes that have metastasis from some other primary cancer.

As used herein, a “tumor” or “neoplastic” or “cancer” cell or tissue means an abnormal cell or tissue exhibiting uncontrolled proliferation and potential to invade other tissues. Non-limiting examples of cancers that can be prevented, treated and/or managed in accordance with the invention include: breast cancer; adrenal cancer; thyroid cancer; pancreatic cancer; pituitary cancers; eye cancers; vaginal cancers; cervical cancers; uterine cancers; ovarian cancers; esophageal cancers; stomach cancers; colon cancers; rectal cancers; liver cancers; gallbladder cancers; cholangiocarcinomas; lung cancers; testicular cancers; prostate cancers; penile cancers; oral cancers; basal cancers; salivary gland cancers; pharynx cancers; skin cancers; kidney cancers; Wilms' tumor; and bladder cancers.

### Methods of Determining Cancer Risk or Prognosis

In accordance with the discoveries detailed herein, the present invention provides several method relating to determining cancer risk or prognosis by detecting CLS. In one embodiment, the method for determining cancer risk or prognosis in a patient comprises detecting the presence of CLS in adipose tissue in a tissue sample from a patient, where the adipose tissue in the sample is in or around an epithelial-derived tissue or organ or the adipose tissue in the sample is associated with a lymph node metastasis arising from an epithelial-derived cancer. In another embodiment, the method for determining cancer risk or prognosis in a patient comprises detecting and quantifying the number of CLS in a sample of adipose tissue from the patient via a CLS index.

One specific embodiment is directed to a method of determining prognosis of a patient with breast cancer which comprises detecting CLS in breast adipose tissue. Another specific embodiment is directed to a method of determining prognosis of a patient with a head and neck carcinoma by detecting CLS in adipose tissue found with lymph node metastasis associated with said carcinoma.

All of these methods involve detecting CLS in tissue sections as described herein using samples that have generally been obtained during routine cancer surgery, during a biopsy to evaluate tissue for the presence of cancer or by other conventional methods of obtaining patient tissue for analysis. In accordance with the present methods, the status of CLS in tissue samples has been found to correlate with worse prognosis or with increased risk when CLS are detected in adipose tissue, as supported by the work described herein in the Examples.

### Methods of Treating Cancer

With an understanding of a cancer patient's CLS status, certain therapeutic treatments are contemplated. Hence the invention provides methods for treating cancer in a patient which comprise determining whether CLS are present in adipose tissue obtained from in or around an epithelial-derived tissue or organ or from adipose tissue associated with a lymph node metastasis arising from an epithelial-derived cancer, and when CLS are detected, and administering a therapeutically-effective dose of a therapeutic agent that reduces the number or function of CLS or the biological consequences of CLS, or any combination thereof.

Other methods of the invention are directed to treating a CLS-related cancer in a patient by administering a Toll-like receptor antagonist, an NF-kB inhibitor, a CDK5 inhibitor, a TNF-α inhibitor, a COX-2 inhibitor, an IL-1 inhibitor, an aromatase inhibitor, a PI3K inhibitor, a Rho-kinase inhibitor, an Akt inhibitor, an mTOR inhibitor, an IGF-1R antagonist, an anti-leptin compound, an AMPK activator, a statin and resveratrol or other polyphenol to the patient for a time and in an amount to improve prognosis, to reduce risk of cancer recurrence, to regress a tumor or to reduce metastasis.

As used herein, “treatment” refers to clinical intervention in an attempt to alter the disease course of the individual or cell being treated, and can be performed either for prophylaxis or during the course of clinical disease. Therapeutic effects of treatment include without limitation, preventing occurrence or recurrence of disease, alleviation of symptoms, diminishment of any direct or indirect pathological consequences of the disease, decreasing the rate of disease progression, amelioration or palliation of the disease state, and remission or improved prognosis. For example, treatment of a cancer patient may result in reduction of tumor size, decrease in the rate of tumor growth, elimination of malignant cells, prevention of metastasis, or the prevention of relapse in a patient whose tumor has regressed.

As used herein, the terms “therapeutically-effective amount” and “effective amount” are used interchangeably to refer to an amount of a composition of the invention that is sufficient to result in the prevention of the development, recurrence, or onset of cancer stem cells or cancer and one or more symptoms thereof, to enhance or improve the prophylactic effect(s) of another therapy, reduce the severity and duration of cancer, ameliorate one or more symptoms of cancer, prevent the advancement of cancer, cause regression of cancer, and/or enhance or improve the therapeutic effect(s) of additional anticancer treatment(s).

A therapeutically-effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

In certain embodiments of the invention, the therapeutically effective amount is an amount that is effective to achieve one, two or three or more of the following results once it is administered: (1) a reduction or elimination of inflammatory markers, such as CLS in adipose tissue; (2) a reduction or elimination in the cancer cell population; (3) a reduction in the growth or size of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) the size of the tumor is maintained and does not increase or increases by less than 10%, or less than 5%, or less than 4%, or less than 2%, (10) an increase in the number of patients in remission, (11) an increase in the length or duration of remission, (12) a decrease in the recurrence rate of cancer, (13) an increase in the time to recurrence of cancer, (14) an amelioration of cancer-related symptoms and/or quality of life and (15) a reduction in drug resistance of the cancer cells.

Therapeutic agents that reduce the number or function of CLS or the biological consequences of CLS, or any combination thereof can be prescribed to a patient identified as CLS-positive. Those of skill in the art can determine appropriate dosages, routes of administration, dosage forms and the like Examples of therapeutic agents that reduces the number or function of CLS or the biological consequences of CLS, or any combination thereof can be prescribed to a patient identified as CLS-positive. Within these agents some act preferentially on macrophages or macrophage functions, e.g., to dampen pro-inflammatory effects, and such agents include as Toll-like receptor antagonists, NF-kB inhibitors, TNF-α inhibitors, COX-2 inhibitors, and IL-1 inhibitors. Other therapeutic agents are effective against hormone-dependent cancers, e.g., aromatase inhibitors. Another group of these agents can reduce the biological consequences of CLS and include such agents as the PI3K inhibitors, Rho-kinase inhibitors, Akt inhibitors, CDK5 inhibitors, mTOR inhibitors, IGF-1R antagonists, anti-leptin compounds and AMPK activators. Finally, statins and resveratrol or other polyphenols may reduce the number of or biological consequences of CLS.

Aromatase inhibitors include aminoglutethimide, anastrozole, exemestane, fadrozole, formestane, letrozole, vorozole, pharmaceutically acceptable salts, prodrugs, and active metabolites thereof.

The therapeutic agent can be administered in monotherapy, in adjunctive or combination therapy with one or more additional pharmacotherapeutic (including chemotherapeutic) agents, in conjunction with radiation therapy, or as adjuvant therapy to a patient undergoing surgery for breast cancer. For example, the agent or aromatase inhibitor can be administered concomitantly with chemotherapy, radiotherapy, and/or surgery to treat the cancer or a secondary tumor derived therefrom.

### Screening Methods

The present invention provides in vitro and in vivo methods for screening test compounds for anti-cancer activity or other medical benefits associated with inhibiting or reducing the number or formation of CLS or a functional consequence of CLS. The in vitro methods take advantage of the fact that macrophages release greater amounts of pro-inflammatory mediators into the medium when stimulated with saturated fatty acids. In one such method, a compound is screened for anti-cancer activity by culturing macrophages with the test compound (or compounds) and a saturated fatty acid in an amount and for a time sufficient to produce conditioned medium comprising pro-inflammatory mediators. The amounts and times can be readily determined by the skilled artisans and examples thereof are provided in the Example section. Thereafter, the medium is harvested and used to culture target cells in the presence of the conditioned medium for a time to induce a cancer-related response to said conditioned medium from those target cells. The target cells useful in the invention include preadipocytes, adipocytes, fibroblasts, epithelial cells and tumor cells, with the choice of cell depending on the cancer-related response to be detected. In detecting cancer-related responses response, one monitors for the inhibition or reduction of that response relative to similarly treated control cultures without said compound. The inhibition or reduction thus indicates the test compound has anti-cancer activity.

Cancer-related responses include inducing aromatase activity, increasing cell proliferation, increasing in cell motility, increasing invasiveness, increasing NFκB activity, or decreasing apoptosis.

In an alternative embodiment, the in vitro method is practiced by culturing the test compound, not with the macrophages, but with the cells that are exposed to the conditioned medium. All variations of this method as described herein are contemplated as part of the invention.

This invention further provides methods of screening candidate compounds using a mouse model of obesity. In one embodiment, a method is provided for screening candidate compounds for ability to treat cancer, comprising identification of compounds that reduce the formation of CLS in a mouse model of obesity, such as an ob/ob mouse, relative to the formation of CLS in ob/ob mice that are not administered the compound. Additional screening methods include, screening for a compound by screening said compound in a dietary model of obesity in mice; determining whether said compound inhibits or reduces formation of CLS in white adipose tissue in the mice. The detection of CLS can be in mammary tissue, in visceral fat, in head or neck tissue or in periprostatic tissue. More particularly, one embodiment for screening a compound for anti-cancer activity is by treating ovary-intact female mice fed a low fat diet and ovariectomized female mice fed a high fat diet with the compound for a time sufficient to assess the effects of the compound; and determining whether the compound preferentially inhibits or reduces formation of CLS-B, or a consequence thereof, in the ovariectomized mice relative to the ovary-intact mice. A compound which inhibits or reduces formation of CLS-B has anti-cancer activity. Similarly, a compound which inhibits or reduces pro-inflammatory mediators, aromatase expression or aromatase activity has anti-cancer activity. Any of the foregoing can be combined with a tumor model, in which the mice are injected with tumor cells, treated with compound and its effects on tumor size, time of arising, CLS formation, pro-inflammatory mediators, aromatase, or other cancer indicator are monitored over time.

The in vivo screening methods of the invention also include methods for determining a compound's activity against CLS by screening a test compound in a dietary or genetic model of obesity in mice; and determining whether the compound inhibits or reduces the number or formation of CLS in breast adipose tissue or a functional consequence of CLS, The model can be adapted by injecting tumor cells and further monitoring the CLS effects and tumor effects of the test compound. In other embodiments, the in vivo methods include methods of screening a compound for a medical benefit using lean and obese mice, with or without a tumor component. After being treated with the test compound, its effects are determined by assessing whether the compound preferentially inhibits or reduces formation of CLS-B or functional consequences of CLS-B in the obese mice relative to the lean mice. The strongest differential effects are usually seen when the lean mouse is an ovary-intact female mouse fed a low fat diet and the obese mouse is an ovariectomized female mouse fed a high fat diet. However, depending on the test purpose, the mice can be male or female and have various diets or be from a genetic model of obesity, provided that one set of mice are lean and the other are obese for whatever reason.

The medical benefits that can be ascertained include but are not limited to anti-cancer activity, anti-diabetogenic activity or anti-obesity activity as these are the types of activities to counter the biological consequences of the presence of CLS in adipose tissue.

The test compounds of the present invention can be any type of compound including small molecules, proteins, peptides, antibodies, siRNA and more and obtained in any manner. The compounds can be tested singly (especially in the in vivo methods) or in combinatorial libraries, especially in the in vitro methods adapted for high through put screening. Combinatorial libraries include biological libraries; peptoid libraries (libraries of molecules having the functionalities of peptides, but with a novel, non-peptide backbone, which are resistant to enzymatic degradation but which nevertheless remain bioactive; see, e.g., Zuckennann 1994); spatially addressable parallel solid phase or solution phase libraries; synthetic library methods requiring deconvolution; the ‘one-bead one-compound’ library method; and synthetic libraries using affinity chromatography selection.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. All references patents, patent applications or other documents cited are herein incorporated by reference in their entirety.

### EXAMPLES

The methods used in the examples are set forth below:

### 1. Animal Models

For the dietary model of obesity, at 5 weeks of age, ovary intact and ovariectomized (OVX) C57BL/6J female mice (Jackson Laboratories) were randomized (n=10/group) to receive either low fat (LF) or high fat (HF) diets. The LF (12450Bi) and HF (D12492i) diets contain 10 kcal % fat and 60 kcal % fat, respectively (Research Diets) and are commonly used in studies of obesity (Hong 2000). Mice were fed these diets ad libitum for 10 weeks before being sacrificed. For the genetic model of obesity, female ob/ob and control C57BL/6J mice were obtained at 8 weeks of age (Jackson Laboratories) and fed PicoLab Rodent Diet 20, #5053 (W.F. Fisher & Son) ad libitum for 3 weeks prior to sacrifice. Following sacrifice, mammary glands and visceral fat were snap frozen in liquid nitrogen and stored at −80° C. for molecular analysis or formalin fixed for histological and immunohistochemical analyses. In experiments to separate the stromal-vascular and adipose fractions of the mammary gland, tissues were directly subjected to cell fractionation. The animal protocol was approved by the Institutional Animal Care and Use Committee at Weill Cornell Medical College.

### 2. Light Microscopy and Immunohistochemistry

Four micron-thick sections were prepared from formalin-fixed, paraffin-embedded mammary gland tissue and visceral fat and stained with hematoxylin and eosin (H+E). The total number of inflammatory foci per section was quantified by a pathologist and the amount of tissue present on each slide was recorded to determine the number of inflammatory foci per cm² of tissue.

Immunohistochemical stains against F4/80 were performed using standard techniques on 4 micron formalin-fixed, paraffin-embedded tissue sections of mammary gland using rat anti-mouse F4/80 primary antibody (Serotec). Detection was done with horseradish peroxidase-labeled goat anti-rat IgG (Jackson ImmunoResearch) and NovaRed substrate (Vector Labs) and counterstains with hematoxylin (Vector Labs).

### 3. Separation of Stromal-Vascular and Adipocyte Fractions

Mammary gland tissue was fractionated into stromal vascular and adipocyte fractions as described (Rodbell 1964). Mammary gland tissue was minced into small pieces and placed in sterile plastic tubes with Krebs-Ringer buffer containing 25 mmol/L NaHCO₃, 11 mmol/L glucose, 25 mmol/L Hepes (pH 7.4), 2% BSA and 1.5 mg/mL collagenase type I. The ratio between adipose tissue mass and incubation solution was 1:4 (w/v). The tissue suspension was incubated at 37° C. with gentle shaking for 45-60 minutes. Once digestion was completed, samples were passed through a sterile 250-μm nylon mesh (VWR). The suspension was centrifuged at 200×g for 10 minutes; the pelleted cells were collected as stromal-vascular fraction (SVF) and the floating cells were considered the adipocyte-enriched fraction. The adipocytes were washed twice with Krebs-Ringer-bicarbonate-Hepes-BSA buffer and centrifuged as above. The SVF was resuspended in erythrocyte lysis buffer consisting of 0.154 mol/L NH₄Cl, 10 mmol/L KHCO₃ and 0.1 mmol/L EDTA, and incubated at room temperature for 10 minutes. The erythrocyte-depleted SVF was centrifuged at 400×g for 5 minutes, the pellet was resuspended and washed four times in Krebs-Ringer-bicarbonate-Hepes-BSA buffer and centrifuged at 400×g for 5 minutes. After washing, the SVF and adipocyte fractions were subjected to analysis or cultured.

### 4. Tissue Culture

Human visceral preadipocytes (ScienCell™) were grown in preadipocyte medium containing 10% FBS.

3T3-L1 cells (ZenBio) were grown in DMEM supplemented with 10% calf bovine serum.

THP-1 cells (ATCC) were maintained in RPMI-1640 medium (Invitrogen) supplemented with 10% FBS. These cells were treated with phorbol 12-myristate 13-acetate (10 ng/mL) overnight to differentiate them into macrophages. Human monocytes (Astarte Biologics) were activated with IFNγ (15 ng/mL) and lipopolysaccharide (15 ng/mL) in RPMI-1640 medium for 4 days. THP-1 cells and blood monocyte-derived macrophages were then treated with saturated fatty acids (Nu-CheK Prep) as indicated. To prepare conditioned medium (CM), the cells were treated with saturated fatty acids for 12 hours in medium comprised of RPMI-1640 and preadipocyte medium (ScienCellTMResearch Laboratories) at a 1:1 ratio. Following treatment with fatty acids, the medium was removed and cells were washed thrice with PBS to remove fatty acids. Subsequently, fresh medium was added for 24 hours. This CM was then collected and centrifuged at 4,000 rpm for 30 minutes to remove cell debris. CM was used to treat preadipocytes.

### 5. Immunoblot Analysis

Lysates were prepared by treating cells with lysis buffer, sonicating for 20 seconds on ice and centrifuging at 10,000×g for 10 minutes to remove particulate material. The protein concentration of the supernatant was measured by the method of Lowry et al. (Lowry 1951). SDS-PAGE was done under reducing conditions on 10% polyacrylamide gels. The resolved proteins were transferred onto nitrocellulose sheets and incubated with primary antisera. Antibodies to phospho-p65, p65, histone H3, Cox-2 and β-actin were used (Santa Cruz Biotechnology). Secondary antibody to IgG conjugated to horseradish peroxidase was used. The blot was probed with the ECL Western blot detection system.

### 6. Northern Blotting

Total RNA was prepared from SVF and adipocytes derived from the mammary gland using an RNA isolation kit. 10 μg of total RNA/lane were electrophoresed in a formaldehyde-containing 1% agarose gel and transferred to nylon-supported membranes. F4/80, aP2 and 18S rRNA probes were labeled with [³²P]dCTP by random priming. The blots were probed as described previously (Kukarni 2001).

### 7. Quantitative Real-Time PCR

Total RNA was isolated using the RNeasy mini kit (Qiagen). For tissue analyses, poly A RNA was prepared with an Oligotex mRNA mini kit (Qiagen). Poly A RNA was reversed transcribed using murine leukemia virus reverse transcriptase and oligo (dT)₁₆ primer. The resulting cDNA was then used for amplification using primers listed Table 1. GAPDH and β-actin were used as endogenous normalization controls for tissue and cell culture analyses, respectively. Real-time PCR was performed using 2×SYBR green PCR master mix on a 7500 Real-time PCR system (Applied Biosystems). Relative fold induction was determined using the ddC_{T} (relative quantification) analysis protocol.

TABLE 1
Amplification Primers
Mouse Primers

| | | | |
|---|---|---|---|
| Cox-2 | Forward: | 5′-ATTCTTTGCCCAGCACTTCA-3′ | (SEQ ID NO. 1) |
| | Reverse: | 5′-GGGATACACCTCTCCACCAA-3′ | (SEQ ID NO. 2) |
| TNF-α | Forward: | 5′-CCAGACCCTCACACTCAGATC-3′ | (SEQ ID NO. 3) |
| | Reverse: | 5′-CACTTGGTGGTTTGCTACGAC-3′ | (SEQ ID NO. 4) |
| IL-1β | Forward: | 5′-TGGGCCTCAAAGGAAAGAAT-3′ | (SEQ ID NO. 5) |
| | Reverse: | 5′-CAGGCTTGTGCTCTGCTTGT-3′ | (SEQ ID NO. 6) |
| Aromatase | Forward: | 5′-AAGCTCTGACGGGCCCTGGT-3′ | (SEQ ID NO. 7) |
| | Reverse: | 5′-ACGTAGCCCGAGGTGTCGGT-3′ | (SEQ ID NO. 8) |
| PR | Forward: | 5′-GGTGGGCCTTCCTAACGAG-3′ | (SEQ ID NO. 9) |
| | Reverse: | 5′-GACCACATCAGGCTCAATGCT-3' | (SEQ ID NO. 10) |
| pS2 | Forward: | 5′-CTGCCCAGGAGAGAAATGAG-3′ | (SEQ ID NO. 11) |
| | Reverse: | 5′-CAGGGTATGAGGGTTCTCCA-3′ | (SEQ ID NO. 12) |
| GAPDH | Forward: | 5′-AATGTGTCCGTCGTGGATCT-3′ | (SEQ ID NO. 13) |
| | Reverse: | 5′-CATCGAAGGTGGAAGAGTGG-3′ | (SEQ ID NO. 14) |

Human Primers

| | | | |
|---|---|---|---|
| Cox-2 | Forward: | 5′-CCCTTGGGTGTCAAAGGTAA-3′ | (SEQ ID NO. 15) |
| | Reverse | 5′-GCCCTCGCTTATGATCTGTC-3′ | (SEQ ID NO. 16) |
| TNF-α | Forward: | 5′-CTGCTGCACTTTGGAGTGAT-3′ | (SEQ ID NO. 17) |
| | Reverse: | 5′-AGATGATCTGACTGCCTGGG-3′ | (SEQ ID NO. 18) |
| IL-1β | Forward: | 5′-GGACAAGCTGAGGAAGATGC-3′ | (SEQ ID NO. 19) |
| | Reverse: | 5′-TCGTTATCCCATGTGTCGAA-3′ | (SEQ ID NO. 20) |
| Aromatase | Forward: | 5′-CACATCCTCAATACCAGGTCC-3' | (SEQ ID NO. 21) |
| | Reverse: | 5′-CAGAGATCCAGACTCGCATG-3′ | (SEQ ID NO. 22) |
| β-actin | Forward: | 5′- AGAAAATCTGGCACCACACC-3′ | (SEQ ID NO. 23) |
| | Reverse: | 5′-AGAGGCGTACAGGGATAGCA-3′ | (SEQ ID NO. 24) |

### 8. Transient Transfections

NF-κB-luciferase (Panomics) and pSVβgal (Promega) were transfected into THP-1 cells using the Amaxa system. After 24 hours of incubation, the medium was replaced with basal medium. The activities of luciferase (BD Biosciences) and β-galactosidase were measured in cellular extracts. For siRNA transfections, THP-1 cells were plated at 60% confluence and transfected with non-targeting siRNA or siRNA targeting TNF-α, IL-1β, Cox-2 and p65 using Dharmafect 4 for 36 hours. Subsequently, the cells were treated with vehicle or saturated fatty acids.

9. ChIP Assay and qPCR

ChIP assays were performed with a kit according to the manufacturer's instructions (Millipore) using 3.5×10⁶ cells crosslinked with 1% formaldehyde. DNA fragments of 200 to 1000-bp were generated and incubated with 1.5 μg of the indicated antibody at 4° C. Immune complexes were precipitated, washed, and eluted as recommended. DNA-protein cross-links were reversed by heating at 65° C. for 4 hours, and the DNA fragments purified and used as a template for PCR amplification.

The following PCR primers were used: TNF-α promoter, forward 5′-GATCCGGAGGAGATTCCTTGA-3′ (SEQ ID NO. 25) and reverse 5′-ACACTCCAGGCACTTAAGGGTCCCGACTCAAGTA-3′ (−255 to −655) (SEQ ID NO. 26); IL-1β promoter, forward 5′-CGTGGGAAAATCCAGTATTTTAATG-3′ (SEQ ID NO. 27) and reverse 5′-CAAATGTATCACCATGCAAATATGC-3′) (−490 to −190) (SEQ ID NO. 28); and Cox-2 promoter forward 5′-AAAGCTATGTATGTATGTGCTGCAT-3′ (SEQ ID NO. 29) and reverse 5′-AACCGAGAGAACCTTCCTTTTTAT-3′)(−7 to −621) (SEQ ID NO. 30). PCR was performed at 94° C. for 30 seconds, 60° C. for 30 seconds, and 72° C. for 45 seconds for 30 cycles. The PCR products generated from the ChIP template were sequenced, and the identity of TNF-α, IL-1β and Cox-2 promoters was confirmed. Real-time PCR was performed as described above.

### 10. Electrophoretic Mobility Shift Assay

Nuclear extracts were prepared from mouse mammary glands using an EMSA kit (Promega). For binding studies, oligonucleotides containing NF-κB sites (Active Motif) were used. The complementary oligonucleotides were annealed in 20 mmol/L Tris (pH 7.6), 50 mmol/L NaCl, 10 mmol/L MgCl₂, and 1 mmol/L dithiothreitol. The annealed oligonucleotide was phosphorylated at the 5′ end with [γ-³²P]ATP and T4 polynucleotide kinase. The binding reaction was performed by incubating 5 μg of nuclear protein in 20 mmol/L HEPES (pH 7.9), 10% glycerol, 300 μg of bovine serum albumin, and 1 μg of poly(dI·dC) in a final volume of 10 μL for 10 minutes at 25° C. The labeled oligonucleotides were added to the reaction mixture and allowed to incubate for an additional 20 minutes at 25° C. The samples were electrophoresed on a 4% nondenaturing polyacrylamide gel. The gel was then dried and subjected to autoradiography at −80° C.

### 11. Aromatase Activity

To determine aromatase activity, microsomes were prepared from cell lysates and tissues by differential centrifugation using established methods (Subbaramaiah 2006). Aromatase activity was quantified by measurement of the tritiated water released from 1β-[³H]-androstenedione (Subbaramaiah 2008). The reaction was also performed in the presence of letrozole, a specific aromatase inhibitor, as a specificity control and without NADPH as a background control. Aromatase activity was normalized to protein concentration.

### 12. PGE₂ Analysis

Levels of PGE₂ in the mammary gland were determined using modified versions of established methods (Blewett 2008, Yang 2006). Approximately 30 mg of frozen tissue was ground to a fine powder with a liquid nitrogen-cooled mortar. Aliquots of 10 μL of internal standard (100 ng/mL), 300 μL of methanol containing 0.25% BHT and 5 μL of formic acid were added to the pulverized tissue. Samples were then sonicated for 3 minutes at 0° C. with an Ultrasonic Processor (Misonix). Following centrifugation, an aliquot (300 μL) of supernatant was mixed with 1.7 mL water and subjected to solid phase extraction. The solution was then applied to an Oasis cartridge (3 cc, 60 mg) (Waters Corp) preconditioned with 2 mL methanol and 2 mL 0.1% formic acid. PGs were eluted with 1.5 mL of methanol followed by 1.5 mL ethyl acetate after the cartridge was washed with 2 mL of 0.1% formic acid. The eluate was then evaporated to dryness under a stream of nitrogen. Samples were reconstituted in 100 μL of methanol/0.1% formic acid (50:50, v:v), before liquid chromatography/tandem mass spectroscopic analysis. Protein concentration was determined by the method of Bradford according to the manufacturer's instructions (Bio-Rad).

LC/MS/MS analyses were performed using a Tandem Quadrupole Mass Spectrometer (Agilent) equipped with an Agilent 1200 binary pump HPLC system using a modified version of the method of Yang 2006. PGs were chromatographically separated using a Gemini 3-μm C6-phenyl 4.6×100 mm analytical column (Phenomenex). The mobile phase consisted of 0.1% formic acid in water (mobile phase A) and 0.1% formic acid in acetonitrile (mobile phase B). The chromatographic baseline resolution for the PGs of interest was achieved using a linear gradient that went from 20% to 40% mobile phase B for 12 minutes and then from 40% to 70% for 8 minutes. This was then increased to 90% mobile phase B over the next 2 minutes and kept at this level for an additional 3 minutes. The flow rate was 0.8 mL/min with a column temperature of 40° C. The mass spectrometer was operated in negative electrospray ionization mode with capillary voltage of 2.9 kV, a cone gas flow rate of 10 L/minute and a shear gas flow rate of 12 mL/minute at 350° C. PGs were detected and quantified using multiple reaction mode monitoring of the transitions m/z 351 to 271 for PGE₂ and m/z 355 to 275 for PGE₂-d₄. The results are expressed as nanograms of PGE₂ per mg of protein. For measurements of PGE₂ in cell culture media, EIA kits were used (Cayman Chemicals).

### 13. Enzyme Immunoassay

TNF-α and IL-1β levels were quantified in cell culture media using EIA kits (R & D systems).

### 14. Statistics

Data generated herein include mouse weights, the number of inflammatory foci, tissue TNF-α, IL-1β, Cox-2 and aromatase mRNA levels and aromatase activity. In general, endpoints that conform to normality assumption, such as the mouse weight data, were summarized in terms of mean±s. d. and compared across multiple groups using ANOVA. Pair-wise comparison of mouse weights were then carried out using Tukey's test to identify pairs of groups with significant difference in weights and adjusted for multiple comparisons. Endpoints that are usually not normally distributed were summarized in terms of median and range and with box-plots graphically. Differences in these endpoints were examined across multiple groups using the non-parametric Kruskal-Wallis test. The Wilcoxon rank sum test was then used for pair-wise comparisons. P-values were adjusted for multiple comparisons using the conservative Bonferroni method. To examine the magnitude of difference between two experimental groups in different fractions of mammary gland tissue, linear mixed-effects models were used to take into account both the within and between mouse variation. Data were log transformed where appropriate to ensure that the underlying normality assumption was satisfied. For in vitro studies, comparisons between groups were made using Student's t-test. A difference between groups of P<0.05 was considered significant.

### Example 1

### Diet-Induced Obesity Causes Inflammation and Elevated Aromatase in Mammary Glands and Visceral Fat

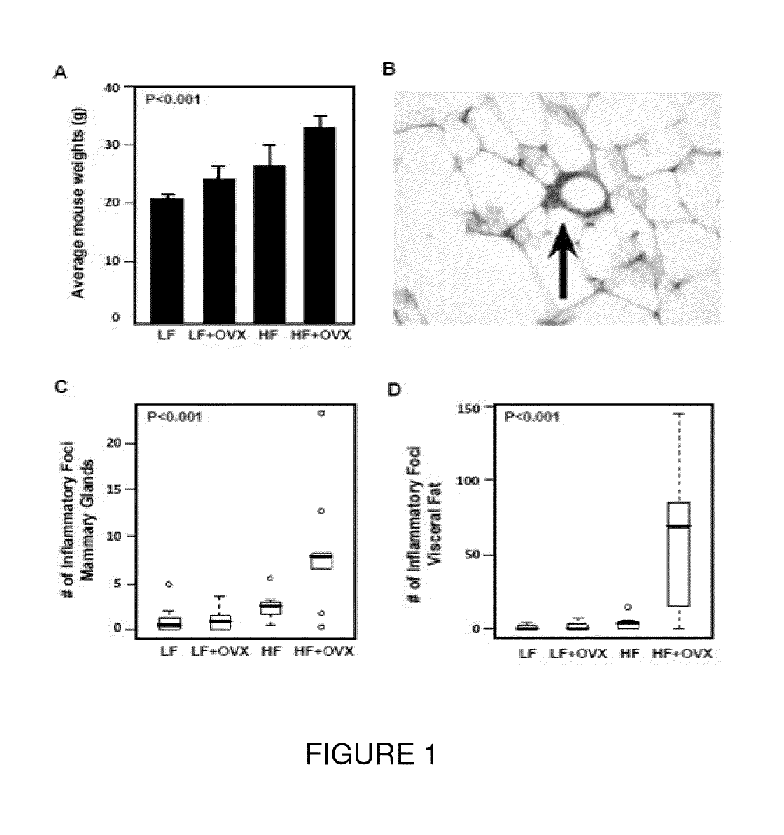

To investigate the effects of dietary fat and ovarian function on weight gain in female mice, ovary-intact and OVX mice (n=10/group) were fed an LF or HF diets for 10 weeks as described herein. Both ovariectomy and feeding a HF diet led to weight gain and the ovariectomized mice fed an HF diet gained the most weight (FIG. 1A). A significant difference in average weights was observed across groups (P<0.001, ANOVA). In particular, OVX mice fed a HF diet had a significantly higher average weight compared to the other groups (P.adj<0.001, Tukey's test).

The mammary glands and visceral fat of the mice were then examined for the presence of inflammatory lesions known as CLS. An example of a CLS-B (in a necrotic adipocyte surrounded by macrophages from mammary tissue stained with H & E) is shown in FIG. 1B. A marked increase in the number of these inflammatory lesions was found in the mammary gland and visceral fat of mice that gained the most weight (FIG. 1, C and D). Significant differences were observed across the four experimental groups for both tissue types (P<0.001, Kruskal-Wallis test). In pair-wise comparisons, OVX mice fed the HF diet showed significantly greater number of inflammatory foci in mammary gland compared with those in the LF diet groups (P.adj=0.01, Wilcoxon rank sum test, P values were adjusted for multiple comparisons with Bonferroni method), and in visceral fat compared with those in the other groups (P.adj<=0.01). To determine whether macrophages were present in these inflammatory lesions, immunohistochemistry was performed to assess F4/80, a macrophage marker. These studies revealed that F4/80 positive cells were abundant in the CLS.

PGE₂ levels were determined in the mammary glands of mice in each of the treatment groups. Significant differences were observed across the four experimental groups (P<0.001, Kruskal-Wallis test). In pair-wise comparisons, OVX mice fed the HF diet showed significantly greater PGE₂ levels compared with those in the LF diet groups (P.adj=0.01, Wilcoxon rank sum test, P values were adjusted for multiple comparisons with Bonferroni method). Consistent with the histological evidence of inflammation, obesity was associated with elevated levels of PGE₂.

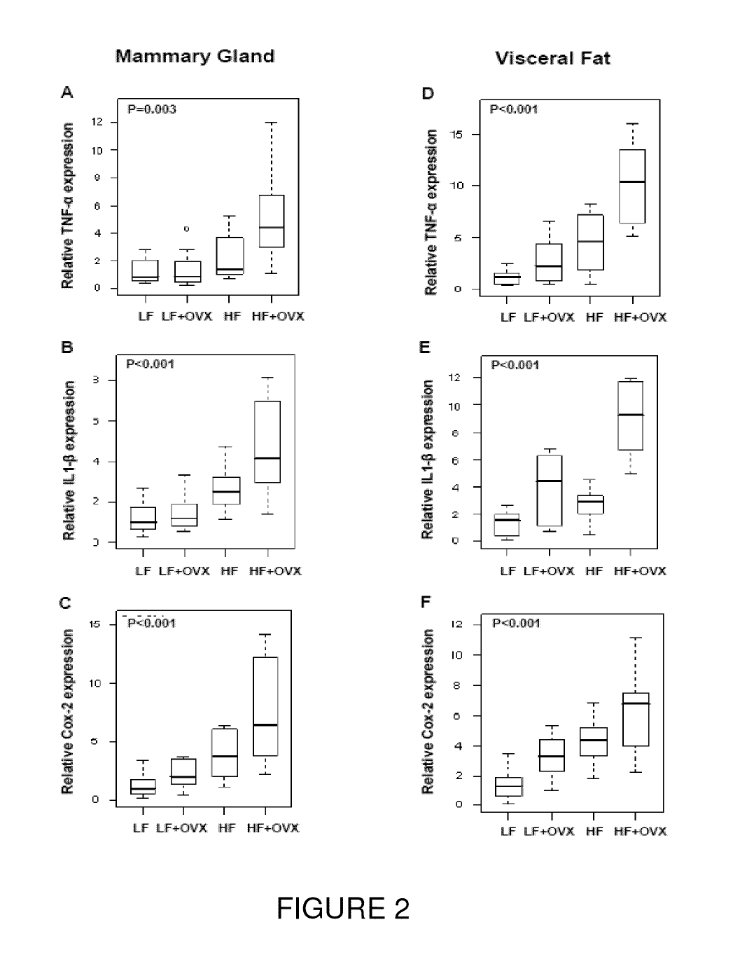

Real-time PCR was used to quantify levels of pro-inflammatory mediators in mammary gland and visceral fat. Obesity-related inflammation was associated with elevated levels of TNF-α, IL-1β and Cox-2 in both the mammary gland and visceral fat. Box-plots of TNF-α, IL-1β and Cox-2 mRNA expression in mammary glands (FIG. 2A-C) and visceral fat (FIG. 2D-F) are shown. Significant differences were observed across the four experimental groups for each pro-inflammatory mediator (P<0.005). In pair-wise comparisons, OVX mice fed a HF diet showed significantly higher expression of the three genes in mammary gland and visceral fat compared with mice in the LF or LF+OVX groups (P.adj<=0.02).

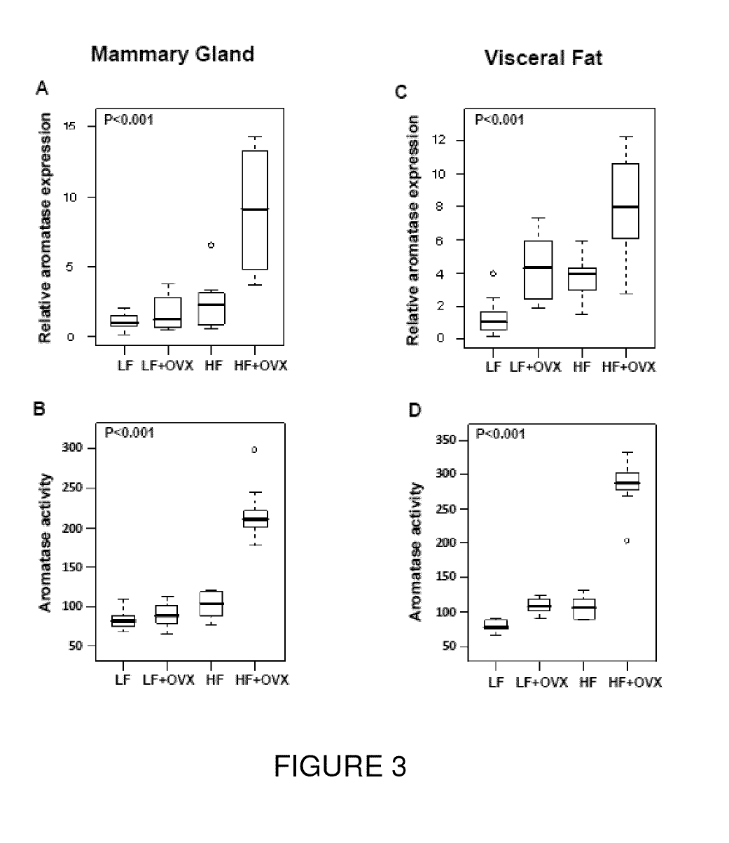

Each of these pro-inflammatory molecules is a known inducer of CYP19 transcription and aromatase activity (Bulun 2005, Brodie 2001, Irahara 2006 Karuppu 2002, Subbaramaiah 2006, Zhao 1996, Zhao 1997, Purohit 2002, hardy 2008, Slama 2009), so aromatase mRNA levels and activity were quantitated in the mammary glands and visceral fat of the different treatment groups. Box-plots of relative aromatase mRNA levels and activity in mammary glands (FIGS. 3A and 3B) and visceral fat (FIGS. 3C and 3D) of mice in each of the four treatment groups are shown. Significant differences were observed across the four experimental groups for aromatase expression and activity (P<0.001). In pair-wise comparisons, OVX mice fed a HF diet had significantly higher levels of aromatase activity (femtomoles/μg protein/hour).

Remarkably, the changes in aromatase expression and activity paralleled the changes in levels of TNF-α, IL-1β and Cox-2 mRNAs in both the mammary gland and visceral fat. Taken together, these data suggest that diet-induced obesity causes inflammation in the mammary gland and visceral fat resulting in increased aromatase expression and activity.

### Example 2

### Genetically-Induced Obesity Causes Inflammation and Elevated Aromatase in Mammary Glands and Visceral Fat

The findings from Example 1 were confirmed in a second obesity model using ob/ob mice. Ob/ob mice are leptin-deficient and have been widely used in studies of obesity. The female ob/ob and control C57BL/6J mice (n=10/group) were obtained at 8 weeks of age and fed PicoLab Rodent Diet 20 ad libitum for 3 weeks prior to sacrifice. At sacrifice, the ob/ob mice weighed 54.3±2.2 gm whereas lean wild-type mice weighed 19.2±0.8 gm (P<0.001). Consistent with the findings in the diet-induced model of obesity, significant increases in the number of inflammatory foci and levels of pro-inflammatory mediators (TNF-α, IL-1β, Cox-2) were observed in the mammary glands and visceral fat of ob/ob vs. wild-type mice (Table 2). Importantly, levels of aromatase mRNA and activity were also significantly increased in both the mammary glands and visceral fat of ob/ob mice (Table 2). Once again, the elevated levels of aromatase paralleled the increased amounts of pro-inflammatory mediators.

TABLE 2
Inflammation and Elevated Aromatase Occurs in Genetic Obesity Model

| | MG | | | VF | | |
|---|---|---|---|---|---|---|
| Endpoint | Wild-type | ob/ob | P | Wild-type | ob/ob | P |
| Inflammatory foci | 0.8 | 13.8 | <0.001 | 0 | 64.5 | <0.001 |
| | (0.0, 2.6) | (8.5, 20.4) | | (0.0, 10.6) | (25.5, 202) | |
| Relative TNF-α | 0.9 | 4.9 | 0.007 | 1.0 | 5.3 | 0.02 |
| expression | (0.6, 4.9) | (0.7, 9.7) | | (0.5, 2.8) | (0.6, 8.7) | |
| Relative IL-1β | 1.0 | 2.9 | 0.02 | 1.1 | 5.7 | 0.005 |
| expression | (0.4, 2.3) | (0.3, 9.7) | | (0.03, 6.0) | (1.1, 7.9) | |
| Relative Cox-2 | 1.0 | 3.8 | 0.001 | 1.0 | 2.2 | 0.02 |
| expression | (0.4, 2.5) | (1.2, 5.7) | | (0.4, 3.7) | (0.6, 4.8) | |
| Relative aromatase | 1.2 | 6.1 | 0.007 | 0.9 | 1.8 | 0.009 |
| expression | (0.2, 5.6) | (0.04, 7.6) | | (0.4, 2.7) | (0.8, 6.8) | |
| Aromatase activity | 90 | 210 | <0.001 | 98 | 272 | <0.001 |
| | (68, 112) | (146, 278) | | (67, 154) | (177, 355) | |

Abbreviations:
Mammary glands (MG);
Visceral fat (VF);
Inflammatory foci, number of inflammatory foci per cm² of tissue;
real-time PCR was used to quantify relative TNF-α, IL-1β, Cox-2 and aromatase transcript levels;
aromatase activity, femtomoles/μg protein/hour.
Values are summarized in median (range), p-values are based on Wilcoxon rank-sum test, n = 10/gp.

### Example 3

### Inflammation and Elevated Aromatase in Mammary Stromal-Vascular and Adipocyte Fractions

The diet-induced obesity model was used to evaluate whether obesity led to increased levels of pro-inflammatory mediators and aromatase in the stromal-vascular fraction (SVF) or adipocyte fractions of the mammary gland, and included mice subjected to OVX because of the link between obesity and increased risk of HR-positive breast cancer in the postmenopausal state. Hence, LF vs. HF+OVX groups fed for 10 weeks were compared since these two groups exhibited the greatest differences in both weight and inflammation (FIG. 1). SVF and adipocyte fractions of the mammary gland were isolated, and to confirm adequacy of the separation, the distribution of F4/80 and aP2, markers of macrophages and adipocytes, respectively was assessed by northern blotting. As expected, F4/80 was expressed in the SVF but not in the adipocyte fraction isolated from the mammary gland. Conversely, aP2 was found in the adipocyte fraction but not in the SVF. Real-time PCR was used to quantify mRNA levels for TNF-α, IL-1β, Cox-2, aromatase, PR and pS2. Interestingly, obesity (HF+OVX vs. LF) was associated with markedly increased levels of TNF-α, IL-1β and Cox-2 mRNAs in the SVF but not in the adipocyte fraction (P<0.001). Increased levels of aromatase mRNA and activity were observed in both SVF and adipocyte fractions in obese mice. Since aromatase is a rate-limiting enzyme for estrogen biosynthesis, the levels of the progesterone receptor (PR) and pS2, prototypic estrogen-inducible genes, were quantitated. Obesity was associated with increased expression of both PR and pS2.

Collectively, the above findings suggest that obesity causes inflammation that is characterized by macrophage-enriched CLS-B in the mammary gland. Elevated levels of pro-inflammatory mediators were found in the SVF.

### Example 4

### SVF Pro-Inflammatory Mediators Induce Aromatase Activity in Adipose Tissue

The pro-inflammatory mediators assessed in the foregoing examples are likely to induce CYP19 transcription and aromatase expression together with aromatase activity in other cell type(s) including adipocytes via a paracrine mechanism.

SVF cells from the mammary glands of mice in the HF+OVX vs. LF groups were cultured overnight in DMEM to quantify the production of pro-inflammatory mediators. Conditioned medium (CM) was then collected and analyzed for levels of TNF-α, IL-1β and PGE₂ by enzyme immunoassay. SVF derived from the HF+OVX group produced markedly increased levels of TNF-α, IL-1β and PGE₂ compared with SVF from the LF group. Consistent with the difference in PGE₂ production, higher levels of Cox-2 protein were found in SVF of the HF+OVX vs. LF group Cox-2 protein as determined by immunoblotting of whole cell lysates.

It was observed that CM derived from the SVF of the HF+OVX vs. LF group led to higher levels of aromatase mRNA and activity in mouse preadipocyte 3T3-LI cells. Experiments were carried out to evaluate the importance of each of the SVF-derived inflammatory mediators (TNF-α, IL-1β and PGE₂) for inducing aromatase. Antibodies were used to neutralize TNF-α and IL-1β in CM-derived from the HF+OVX mammary gland SVF. Briefly, CM from HF+OVX SVF was incubated with control IgG, TNF-α IgG or IL-1β IgG overnight at 4° C. to neutralize TNF-α and IL-1β. 3T3-L1 cells were then treated for 24 hours prior to measurements of relative aromatase mRNA levels and aromatase activity.

Neutralizing either TNF-α or IL-1β attenuated CM-mediated induction of aromatase in 3T3-L1 cells. Additionally, treatment of macrophage-enriched SVF with celecoxib (5 μmol/L), a selective Cox-2 inhibitor, blocked the release of PGE₂ into the CM. Following Cox-2 inhibition, the ability of SVF-derived CM to induce aromatase mRNA and aromatase activity in 3T3-L1 cells was markedly attenuated. Taken together, these data suggest that SVF isolated from the mammary gland of obese vs. lean mice produces increased levels of TNF-α, IL-1β and Cox-2-derived PGE₂, each of which contributes, in turn, to the induction of aromatase in preadipocytes.

### Example 5

### Saturated Fatty Acids Stimulate the Production of Pro-Inflammatory Mediators

Experiments were carried out in vitro to determine if saturated fatty acids stimulated the production of pro-inflammatory molecules by macrophages leading, in turn, to elevated aromatase expression in preadipocytes. Saturated fatty acids ranging in chain length from C12 to C18 including lauric acid (LA), myristic acid (MA), palmitic acid (PA) and stearic acid (SA) were used at concentrations of 0, 2.5, 5 and 10 μmol/L (n=6). Real-time PCR was used to quantify TNF-α, IL-1β and Cox-2 mRNAs. Levels of TNF-α protein, IL-1β protein and PGE₂ in the culture medium were determined by enzyme immunoassay. Cox-2 protein abundance was determined by immunoblotting of whole cell lysates. β-actin was used as a loading control.

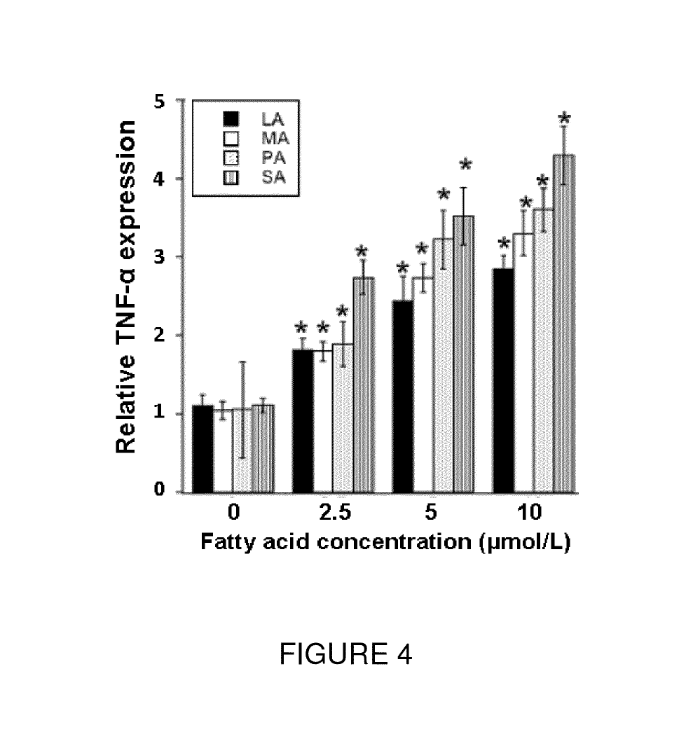

Treatment with each of these saturated fatty acids caused dose-dependent induction of TNF-α, IL-1β, and Cox-2 in THP-1 cells, a cell line with properties of human monocyte-derived macrophages. An example of these effects is shown in FIG. 4 for TNF-α. Induction of Cox-2 mRNA by saturated fatty acids was associated with a corresponding increase in Cox-2 protein and PGE₂ production. Using the same treatment protocol, saturated fatty acids also induced pro-inflammatory mediators in human blood monocyte-derived macrophages.

Further, to investigate whether conditioned medium from saturated fatty acid-treated macrophages induced aromatase in preadipocytes, THP-1 cells or human blood monocyte-derived macrophages were treated with 0, 2.5, 5 or 10 μmol/L LA, MA, PA or SA as described above to generate CM (n=6). This CM was used to treat preadipocytes for 24 hours. Aromatase mRNA and activity levels were determined. Treatment with CM derived from either saturated fatty acid-treated THP-1 cells or human blood monocyte-derived macrophages induced aromatase mRNA and activity in human preadipocytes. Neutralizing either TNF-α or IL-1β by incubating the CM from SA-treated cells with neutralizing antibodies to TNF-α, IL-1β or control IgG overnight at 4° C. attenuated CM-mediated induction of aromatase in preadipocytes.

Next, THP-1 cells were transfected as indicated with control siRNA or TNF-α siRNA, IL-1β siRNA or Cox-2 siRNA for 36 hours. Cells were then treated with vehicle (control) or 10 μmol/L SA as described above. The levels of TNFα, IL-1β and PGE₂ were measured by enzyme immunoassay in the CM. Cox-2 protein abundance was determined by immunoblotting of whole cell lysates using β-actin was used as a loading control. Preadipocytes were treated with THP-1 cell-derived CM for 24 hours prior to measurements of aromatase mRNA and activity. THP-1 cells were also treated with vehicle, SA or SA and 5 μmol/L celecoxib for 12 hours. Following treatment, the medium was removed and cells were washed. Subsequently, fresh medium was added for 24 hours to generate CM. PGE₂ production was measured in CM. Preadipocytes were treated with THP-1 cell-derived CM for 24 hours prior to measurements of aromatase mRNA and activity.

Use of siRNA to silence either TNF-α or IL-1β in THP-1 cells led to similar suppressive effects. Additionally, either silencing of Cox-2 or treatment of THP-1 cells with celecoxib blocked saturated fatty acid-mediated induction of PGE₂ release into the CM. Following Cox-2 inhibition, the ability of macrophage-derived CM to induce aromatase mRNA or aromatase activity in preadipocytes was attenuated. Taken together, these data suggest that saturated fatty acid-mediated induction of TNF-α, IL-1β and Cox-2 in macrophages contributes to the induction of aromatase in preadipocytes.

### Example 6

### Activation of NF-κB Contributes to Increased Aromatase Levels in Obesity

To investigate the potential role of the transcription factor NF-κB in saturated fatty acid-mediated induction of TNF-α, IL-1β and Cox-2 in macrophages, transient transfection assays were performed in saturated fatty acid-treated THP-1 cells with an NF-κB-luciferase reporter construct and pSV-βgal constructs. Cells were treated with 10 μmol/L LA, MA, PA or SA for 24 hours and luciferease and β-galactosidase activity was measured. Saturated fatty acids induced NF-κB-luciferase activity in THP-1 cells.

DNA-protein binding complexes to NF-κB binding sites were examined by electrophoretic mobility shift assays (EMSA). Nuclear protein was prepared from THP-1 cells treated with 0, 2.5, 5 or 10 μmol/L of SA for 1 hour. In another experiment, nuclear protein was prepared from THP-1 cells treated with vehicle or 10 μmol/L SA for 1 hour, and the nuclear protein was incubated with normal IgG or different concentrations of antibodies to p65. In each case, 10 μg of nuclear protein was incubated with a ³²P-labeled oligonucleotide containing NF-κB binding sites. The protein-DNA complexes that formed were separated on a 4% polyacrylamide gel. EMSA indicated that saturated fatty acids stimulated binding of nuclear protein to a ³²P-labeled NF-κB consensus sequence which could be abrogated when a large excess of cold probe was used. Supershift assays indicated that p65, a component of NF-κB, was present in the binding complex.

THP-1 cells were treated as indicated with 0, 2.5, 5 or 10 μmol/L LA, MA, PA or SA for 30 minutes and the abundance of phospho-p65 and p65 protein in cell lysates was determined by immunoblotting. The abundance of phospho-p65 was determined by immunoblotting in cytosolic and nuclear preparations of cells treated with vehicle (control) or 10 μmol/L SA for 30 minutes. β-actin and histone H3 were used as cytosolic and nuclear markers, respectively. Consistent with the activation of NF-κB, saturated fatty acids stimulated the phosphorylation of p65 and its translocation from cytosol to nucleus.

ChIP assays were performed to evaluate the potential role of NF-κB in regulating the expression of TNF-α, IL-1β and Cox-2 in macrophages by determining if saturated fatty acids stimulated the binding of p65 to the promoters of each of these pro-inflammatory genes. THP-1 cells were treated with vehicle (control) or 10 μmol/L LA, MA, PA or SA for 3 hours. Chromatin fragments were immunoprecipitated with antibodies against phospho-p65 and the TNF-α, IL-1β and Cox-2 promoters were amplified by real-time PCR. DNA sequencing was carried out, and the PCR products were confirmed to be the correct promoters. These promoters were not detected when normal IgG was used or when antibody was omitted from the immunoprecipitation step.

To further evaluate the importance of p65 in regulating the production of both pro-inflammatory mediators and the induction of aromatase, THP-1 cells were treated with control siRNA or an siRNA to p65. The abundance of p65 protein was determined by immunoblotting in cell lysates. Further, THP-1 cells were untransfected, or transfected with control siRNA or p65 siRNA and subsequently treated with 0 or 10 μmol/L LA, MA, PA or SA to generate CM as described above. Enzyme immunoassays were used to quantify levels of TNF-α, IL-1β and PGE₂ in the CM. Preadipocytes were treated with THP-1 cell-derived CM for 24 hours prior to measurements of aromatase mRNA and activity. Silencing of p65 inhibited the production of pro-inflammatory mediators (TNF-α, IL-1β, PGE₂) in response to treatment with saturated fatty acids. Silencing of p65 in THP-1 cells also suppressed CM-mediated induction of aromatase expression and activity in preadipocytes.

The effects of BAY11-7082, a pharmacological inhibitor of NF-κB, were evaluated. THP-1 cells were transiently transfected with NF-κB-luciferase and pSV-βgal constructs and cells were subsequently treated with vehicle, 10 μmol/L SA or SA plus 0, 5 or 10 μmol/L of BAY11-7082 (InvivoGen) for 12 hours. NF-κB luciferase activity was normalized to β-galactosidase activity. Additionally, THP-1 cells were treated with vehicle (control), 10 μmol/L SA or SA plus 10 μmol/L BAY11-7082 for 24 hours. Levels of TNF-α protein, IL-1β protein and PGE₂ in the CM were determined by enzyme immunoassay. Preadipocytes were treated with THP-1 cell-derived CM for 24 hours prior to measurements of aromatase mRNA and activity. Treatment of THP-1 cells with BAY11-7082 (InvivoGen) suppressed both SA-mediated activation of NF-κB and the production of increased amounts of TNF-α, IL-1β and PGE₂. Following NF-κB inhibition, the ability of macrophage-derived CM to induce aromatase expression and activity was suppressed.

Because of the role that NF-κB was found to play in regulating pro-inflammatory mediator production and aromatase expression in vitro, complementary studies were carried out on tissues from the diet-induced model of obesity. EMSAs were performed as described above with nuclear extracts isolated from mammary gland tissue in the four treatment groups described in Example 1. Consistent with the elevated levels of TNF-α, IL-1β and Cox-2 in mammary glands of obese mice (FIG. 2), NF-κB binding activity was increased in this group and p65 was found in the binding complex. Using the methods and conditions of Example 1, higher levels of TNF-α, IL-1β and Cox-2 message were found in the SVF vs. adipocyte fraction of mammary glands derived from obese mice. Notably, higher levels of NF-κB binding activity were also found in the SVF vs. adipocyte fractions prepared from the mammary glands of obese mice. Consistent with the in vitro findings, supershift assays indicated that p65 was present in the binding complex. Taken together, these findings suggest that NF-κB plays a key role in regulating the production of pro-inflammatory mediators in macrophages leading, in turn, to the induction of aromatase in the adipose fraction of the mammary gland.

### Example 7

### CLS-B are Found in Human Breast White Adipose Tissue

### 1. Study Population and Samples for Examples 7-9

Women undergoing mastectomy (but not lumpectomy) were consented under a standard tissue acquisition IRB-approved protocol. The sample size of 30 women ensured inclusion of women with a range of BMIs. The menopausal status and BRCA1/2 mutation status of each patient was ascertained when available as well as height and weight measurements to calculate BMI. For one subject, BMI was calculated based on self-reported measurements of height and weight. Standard definitions of normal, overweight and obese were used: normal, BMI 18.5-24.9; overweight, BMI 25-29.9; obese, BMI≧30.

For each of the 30 study subjects, standard pathological processing was carried out. Additional paraffin blocks were produced at random from normal breast tissue. The first patient had two tissue blocks (each approximately 1.2 g; 2 cm×1 cm×0.3 cm). Of the remaining 29 cases, all had five blocks with the exception of the third case which had four blocks. From each available tissue block, two sections with a thickness of five microns were cut. The first section was stained using H+E for morphologic assessment. The second section was stained for a macrophage marker, CD68 (mouse monoclonal KP1 antibody, Dako; dilution 1:4000). All cases were reviewed by a dedicated breast histopathologist. Light microscopy was used to assess for evidence of CLS-B.

### 2. Statistical Analyses for Examples 7-9

The primary endpoints of the study included CLS-B positivity, defined as the presence or absence of CLS-B in any of the sections stained for CD68, and the CLS-B intensity, defined as the percent of blocks with positive CD68 staining for each case. Baseline patient characteristics including age, BMI, menopausal status, and BRCA mutation status were recorded. Average adipocyte size, aromatase mRNA levels, and aromatase activity were also obtained for each case. The association between CLS-B positivity and each baseline patient characteristic including BMI (both as a continuous variable and a categorical variable) was examined using logistic regression and Fisher's exact test where appropriate. The association between CLS-B index and BMI and between CLS-B index and average adipocyte size was evaluated using logistic regression. Strength of correlation between BMI and adipocyte size, between CLS-B index and the levels of aromatase, and between BMI and aromatase were quantified using the Spearman's rank correlation coefficient. Correlation coefficients were tested against the null hypothesis that the correlation coefficients being 0. Results with P values less than 0.05 were considered statistically significant. Correlation was considered as strong, moderate, or weak if the correlation coefficient was 0.75 or more, 0.45 or more, and less than 0.75, or less than 0.45, respectively.

### 3. Results

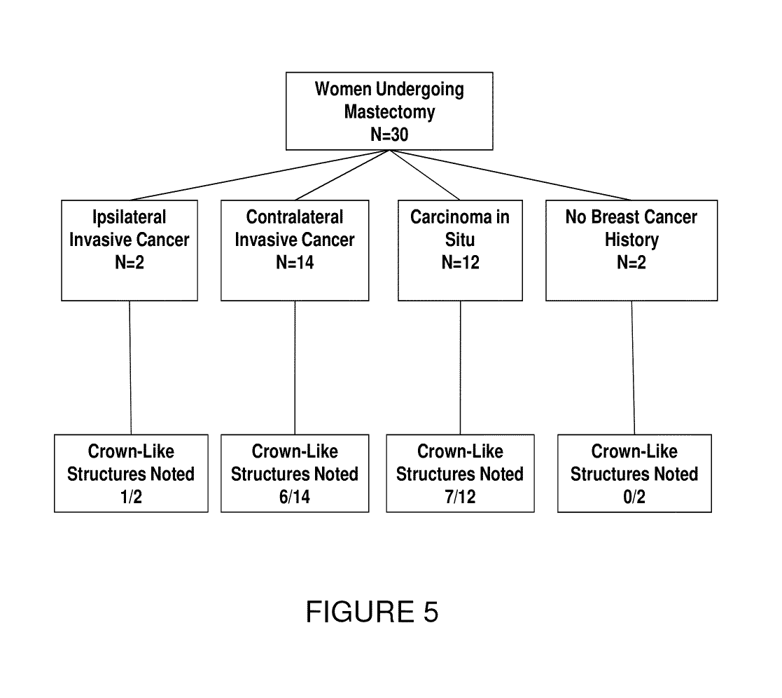

The study consort is diagrammed in FIG. 5 and the baseline characteristics are shown in Table 3. The 30 enrollees had a median age of 50 (range 26-70). Twenty eight of the 30 had no evidence of invasive cancer whereas two had ipsilateral invasive duct cancer (FIG. 5). Seven of the 30 patients who underwent surgery were known carriers of mutations in either the BRCA1 or BRCA2 genes.

On H+E examination of breast white adipose tissue, seven (23%) patients had evidence of CLS-B (FIG. 6). Immunohistochemical staining for CD68, a macrophage marker, revealed CLS-B in 14 of 30 (47%) cases (FIG. 7). All tissue sections that were CLS-B positive by H+E were also CD68 positive. Given the higher sensitivity of detecting CLS-B using CD68 immunohistochemistry compared with H+E, subsequent analyses were based on results obtained with CD68 immunohistochemistry. Notably, CLS-B was identified in both pre-menopausal and post-menopausal women as well as in two of the seven BRCA mutation carriers.

TABLE 3
Baseline Characteristics of Patients

| | Overall | No Evidenc of | CLS-B showed | |
|---|---|---|---|---|
| Characteristic | (n = 30) | CLS-B (n = 16) | (n = 14) | P |
| Age, y | | | | |
| Mean ± SD | 49.9 ± 10.4 | 48.6 ± 11.8 | 51.4 ± 8.7 | 0.46 |
| Median (range) | 50 (26-70) | 48.5 (26-70) | 51.5 (38-68) | |
| Menopausal status, n (%) | | | | |
| Pre | 16 (53) | 9 | 7 | 1 |
| Post | 14 (47) | 7 | 7 | |
| BMI | | | | |
| Mean ± SD | | 24.5 ± 4.7 | 31.6 ± 6.4 | 0.01 |
| Median (range) | | 22.5 (19.3-35.7) | 29.2 (22.1-45.6) | |
| BRCA status, n (%) | | | | |
| Known mutation | 7 (23) | 5 | 2 | 1.0^{a} |
| No known mutation | 9 (30) | 6 | 3 | |
| Unknown (not tested) | 14 (47) | 5 | 9 | |
| Breast surgery | | | | |
| Ipsilateral breast cancer | 2 (7) | 1 | 1 | 0.68 |
| Contralateral breast | 14 (47) | 8 | 6 | |
| cancer | | | | |
| Carcinoma in situ | 12 (40) | 5 | 7 | |
| No breast cancer history | 2 (7) | 2 | 0 | |

^{a}Compared among those with known BRCA status.

### Example 8

### Presence and Intensity of CLS-B are Associated with BMI

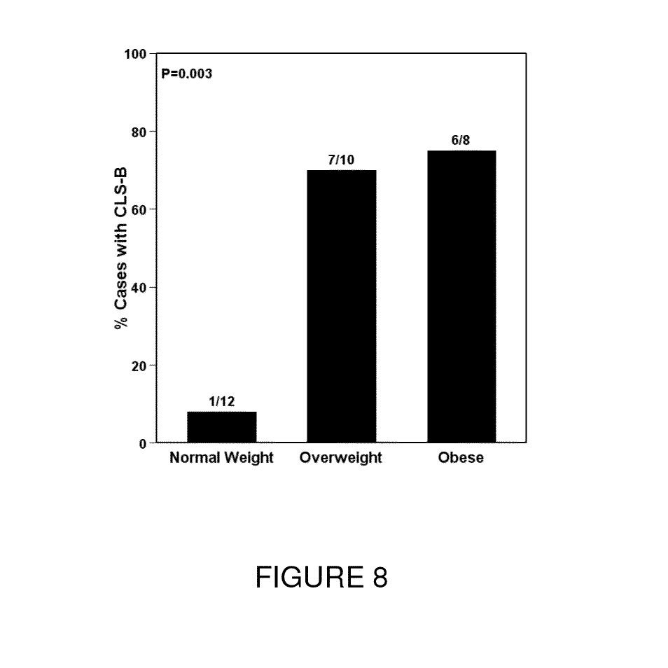

To evaluate the association between BMI and CLS-B, the percentage of cases positive for CLS-B was compared among patients who were normal BMI, overweight or obese in the study population from Example 7. Increasing likelihood of CLS-B positivity was associated with increasing BMI. Specifically, CLS-B were observed in seven out of ten (70%) overweight and six out of eight (75%) obese patients, while only one in 12 (8%) normal weight patients had evidence of CLS-B (P=0.003, FIG. 8).

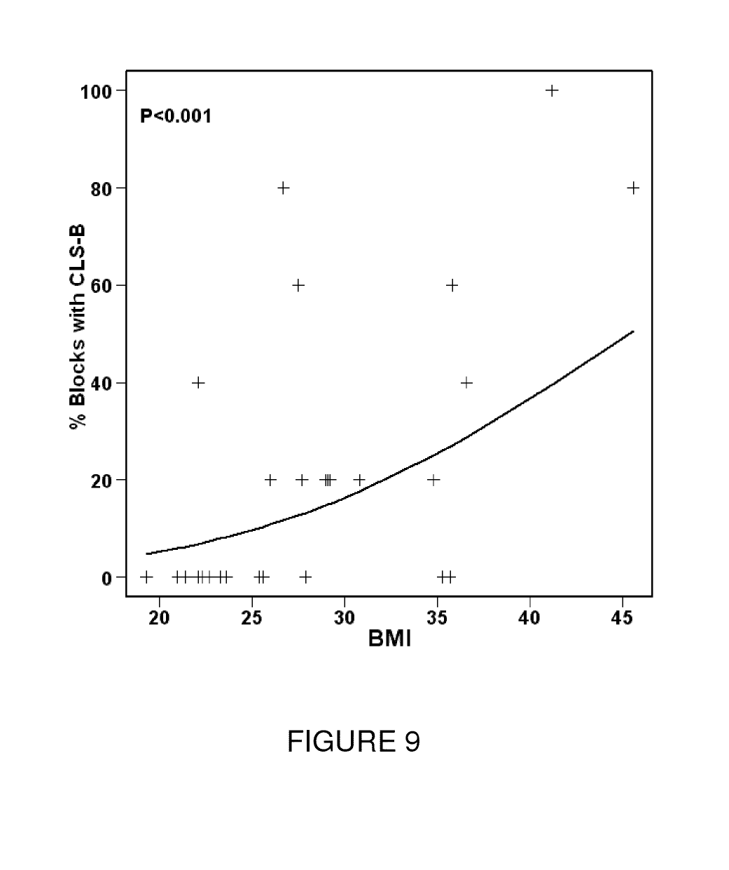

The severity of inflammation (CLS-B intensity) also varied according to BMI. Of the 14 CLS-B positive cases, seven patients had evidence of CLS-B in one tissue block, two patients had CLS-B in 2/5 blocks, two patients had CLS-B in 3/5 blocks, two patients had CLS-B in 4/5 blocks and one patient had CLS-B in 5/5 blocks. Increasing intensity of CLS-B (% of blocks with CLS-B) was associated with increasing BMI in logistic regression analysis (P<0.001) (FIG. 9).

In human subcutaneous and visceral fat, increased adipocytes size has been associated with adipocyte cell death and the presence of CLS (Cinti 2005). Given this background, the relationship between BMI and adipocyte diameter in the breast was determined.

To determine adipocyte diameter, breast biopsies were photographed at 20×, utilizing an Olympus BX50 microscope and MicroFire digital camera (Optronics). Images were stored in the tagged image file format (TIFF) and adipocyte size determined utilizing the Linear Dimensional Tool (LDT) in Canvas 11 (ACS Systems of America, Inc.). The LDT was calibrated utilizing a 20× photomicrograph of a stage micrometer etched with 10-mm divisions. Each photomicrograph of breast adipose tissue was bisected by a vertical and horizontal line. The maximum diameter of adipocytes that fell on the vertical and horizontal lines was determined utilizing the LDT. In this manner, adipocytes were randomly selected for measurement. Two histologic slides were prepared for each biopsy. A total of 18 to 42 individual cells were measured for each patient. Photomicrographs and measurement of adipocytes were conducted blind to patient identity. To determine the reproducibility of these measurements, two regions from 9 biopsies were photographed and the average adipocyte diameters were compared. The mean diameters of adipocytes measured in different fields from the same patient were highly correlated (r=0.95; P<0.001).

A positive correlation was observed between BMI and adipocyte size (P<0.001). Notably, increasing adipocyte size was associated with a statistically significant increase in the CLS-B index (P=0.01).

### Example 9

### Breast Inflammation is Associated with Increased Aromatase Expression and Activity

Levels of aromatase mRNA and activity were determined in breast tissue from each of the 30 subjects, as generally described under EXAMPLES Sections 7 and 11, and correlated with BMI and CLS-B index. Both elevated BMI and breast inflammation were associated with increased amounts of aromatase mRNA and activity. Levels of aromatase mRNA correlated with both BMI (r=0.42, P=0.02) and CLS-B index (r=0.75, P<0.001), but the correlation was stronger with CLS-B index. Similarly, aromatase activity correlated more strongly with CLS-B index (r=0.88, P<0.001) than BMI (r=0.5, P=0.02).

Activation of NF-kB, a transcription factor implicated in obesity-related inflammation (Olefsky 2010, Subbaramaiah 2011), stimulates the production of several proinflammatory mediators that can induce aromatase. Hence, EMSA was carried out as generally described under EXAMPLES section 10 to determine NF-kB binding activity, using nuclear protein from breast samples with or without CLS-B. Breast samples containing CLS-B were from overweight or obese women. In these studies, NF-kB binding activity was higher in samples containing CLS-B. Supershift assays indicated that p65 was present in the binding complex.

### Example 10

### CLS are Observed in Periprostatic Tissue in Genetic Obesity Model

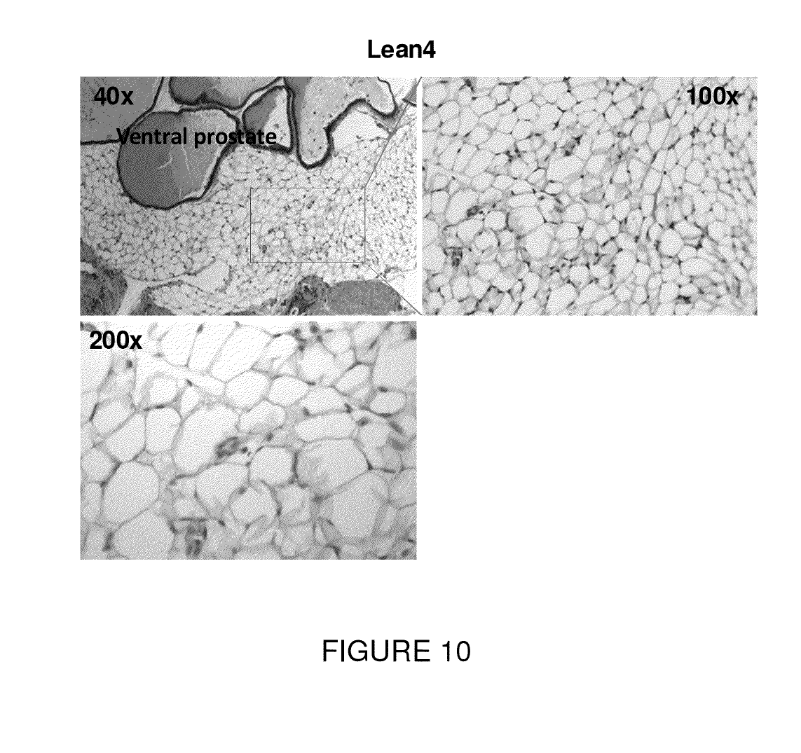

Prostate tissue, like mammary gland tissue, is an epithelial-derived tissue which interfaces with adipose cells. Male ob/ob and control mice were obtained and fed as described in Example 2. Following sacrifice at 11 weeks of age, prostate tissue and adjacent fat was formalin fixed for histological analyses. FIG. 10 shows H+E staining of prostate and adjacent adipose tissue from lean mice (control), and FIG. 11 shows H+E staining of periprostatic adipose tissue adjacent adipose tissue from obese mice. The arrows in the 100× and 200× magnification panels indicate the presence of CLS in the periprostatic tissue, termed here CLS-P. Thus significant macrophage infiltration was present in the periprostatic tissue of ob/ob mice, while significant macrophage infiltration was absent in control mice.

### Example 11

### CLS are Observed in Neck Tissue in Diet-Induced Obesity Model

Five week old female mice, ovary-intact (n=10 per group), and OVX mice (OVX at 4 weeks of age, n=10 per group) were fed an LF or HF diet for 10 weeks as described herein. Mice were sacrificed and neck tissues were prepared and stained with H+E. CLS were observed in the white adipose tissue, indicating significant macrophage infiltration was present in the HF mice, and absent in the LF mice.

### Example 12

### CLS-N are Found in Human Neck Adipose Tissue

The characteristics of the study population are provided in Table 4. This population consisted of 27 cases of oral squamous cell carcinoma (T1, T2) in which neck dissections had been performed. Tissue sections were stained with H+E and with anti-CD68 antibodies as described herein to identify CLS structures (CLS-N). CLS-N was observed in peri-nodal fat and macrophages containing phagocytosed fat were readily observed. In two patients, CLS were found within metastatic squamous cell carcinoma.

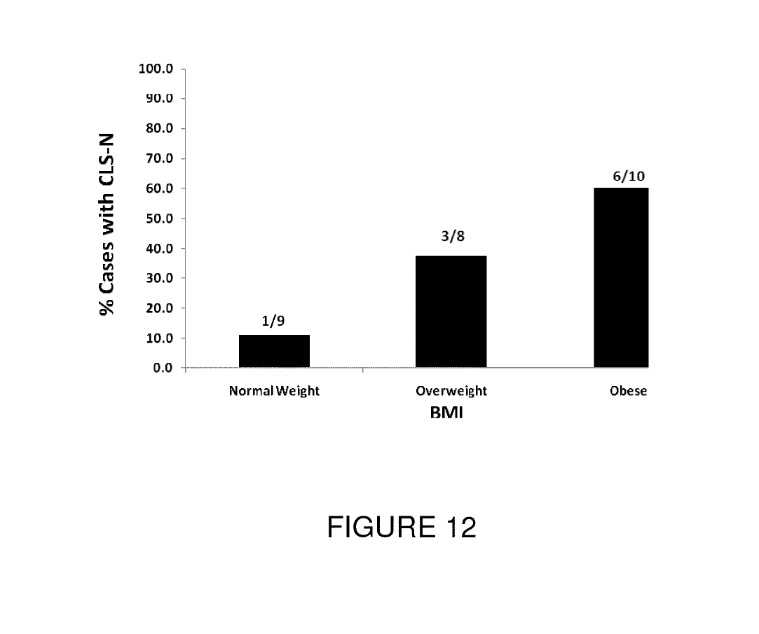

A pathologist unaware of the subject's BMI scored all cases for CLS-N. The percentage of cases with CLS-N is plotted as a function of BMI (FIG. 12). The percentage of cases with CLS-N correlated with BMI such that increasing BMI was associated with increasing likelihood of observing CLS-N. Further, the presence of CLS-N was more prevalent patients with lymph node metastases, with 1/12 patients with lymph nodes negative for metastasis showing CLS-N and 9/15 patients with lymph-node positive disease showing CLS-N.

Hence, CLS-N were found in humans and correlated with BMI as well as with lymph node metastasis suggesting that CLS-N status can used as a prognostic biomarker. The finding of CLS within lymph-node metastases is consistent with macrophage-derived factors promoting tumor progression.

TABLE 4
Baseline Characteristics of Patients

| | Overall | | No evidence of | | CLS-N showed | |
|---|---|---|---|---|---|---|
| Characteristic | (n = 27) | | CLS-N (n = 17) | | (n = 10) | |
| Gender | | | | | | |
| Male | 15 | (55.6) | 11 | (64.7) | 4 | (40.0) |
| Female | 12 | (44.4) | 6 | (35.3) | 6 | (60.0) |
| Age, y | | | | | | |
| Mean ± SD | 56.5 ± 9.9 | | 55.1 ± 7.9 | | 58.9 ± 12.7 | |
| Median (range) | 58 | (37-75) | 53 | (43-75) | 60 | (37-75) |
| BMI | | | | | | |
| Mean ± SD | | | 28.0 ± 7.1 | | 33.2 ± 6.4 | |
| Median (range) | | | 26.1 | (20.3-43.7) | 34.7 | (24.1-46.7) |
| Tobacco | | | | | | |
| History, n (%) | | | | | | |
| Current | 6 | (22.2) | 4 | (23.5) | 2 | (20.0) |
| Discontinued | 12 | (44.4) | 8 | (47.1) | 4 | (40.0) |
| Never | 9 | (33.3) | 5 | (29.4) | 4 | (40.0) |
| ETOH History, | | | | | | |
| n (%) | | | | | | |
| Current | 18 | (66.7) | 12 | (70.6) | 6 | (60.0) |
| Discontinued | 3 | (11.1) | 1 | (5.9) | 2 | (20.0) |
| Never | 6 | (22.2) | 4 | (23.5) | 2 | (20.0) |
| T stage, n (%) | | | | | | |
| T1 (≦2 cm) | 18 | (66.7) | 12 | (70.6) | 6 | (60.0) |
| T2 (>2 and | 9 | (33.3) | 5 | (29.4) | 4 | (40.0) |
| ≦4 cm) | | | | | | |
| LN Metastasis, | 15 | (55.6) | 6 | (35.3) | 9 | (90.0) |
| n (%) | | | | | | |
| LN Metastasis | 7 | (46.7) | 2 | (33.3) | 5 | (55.5) |
| with | | | | | | |
| Extracapsular | | | | | | |
| Extension, | | | | | | |
| n (%) | | | | | | |

### REFERENCES

The references cited herein are listed below:
Bachelot T, Ray-Coquard I, Menetrier-Caux C, Rastkha M, Duc A, Blay J Y. Prognostic value of serum levels of interleukin 6 and of serum and plasma levels of vascular endothelial growth factor in hormone-refractory metastatic breast cancer patients. Br J. Cancer. 2003; 88:1721-6.
Blewett A J, Varma D, Gilles T, Libonati J R, Jansen S A. Development and validation of a high-performance liquid chromatography-electrospray mass spectrometry method for the simultaneous determination of 23 eicosanoids. J Pharm Biomed Anal. 2008; 46:653-62.
Brodie A M, Lu Q, Long B J, Fulton A, Chen T, Macpherson N, et al. Aromatase and COX-2 expression in human breast cancers. J Steroid Biochem Mol Biol. 2001; 79:41-7.
Bulun S E, Lin Z, Imir G, Amin S, Demura M, Yilmaz B, et al. Regulation of aromatase expression in estrogen-responsive breast and uterine disease: from bench to treatment. Pharmacol Rev. 2005; 57:359-83.
Calle E E, Kaaks R. Overweight, obesity and cancer: epidemiological evidence and proposed mechanisms. Nat Rev Cancer. 2004; 4:579-91.
Calle E E, Rodriguez C, Walker-Thurmond K, Thun M J. Overweight, obesity, and mortality from cancer in a prospectively studied cohort of U.S. adults. N Engl J. Med. 2003; 348:1625-38.
Cancello R, Henegar C, Viguerie N, Taleb S, Poitou C, Rouault C, et al. Reduction of macrophage infiltration and chemoattractant gene expression changes in white adipose tissue of morbidly obese subjects after surgery-induced weight loss. Diabetes. 2005; 54:2277-86.
Cinti S, Mitchell G, Barbatelli G, Murano I, Ceresi E, Faloia E, et al. Adipocyte death defines macrophage localization and function in adipose tissue of obese mice and humans. J Lipid Res. 2005; 46:2347-55.
Cleary M P, Grossmann M E. Minireview: Obesity and breast cancer: the estrogen connection. Endocrinology. 2009; 150:2537-42.
Dandona P, Weinstock R, Thusu K, Abdel-Rahman E, Aljada A, Wadden T. Tumor necrosis factor-alpha in sera of obese patients: fall with weight loss. J Clin Endocrinol Metab. 1998; 83:2907-10.
Duggan C, Irwin M L, Xiao L, Henderson K D, Smith A W, Baumgartner R N, et al. Associations of insulin resistance and adiponectin with mortality in women with breast cancer. J Clin Oncol. 2011; 29:32-9.
Ewertz M, Jensen M B, Gunnarsdottir K A, Hojris I, Jakobsen E H, Nielsen D, et al. Effect of obesity on prognosis after early-stage breast cancer. J Clin Oncol. 2011; 29:25-31.
Goodwin P J, Ennis M, Pritchard K I, Trudeau M E, Koo J, Madarnas Y, et al. Fasting insulin and outcome in early-stage breast cancer: results of a prospective cohort study. J Clin Oncol. 2002; 20:42-51.
Hardy D B, Janowski B A, Chen C C, Mendelson C R. Progesterone receptor inhibits aromatase and inflammatory response pathways in breast cancer cells via ligand-dependent and ligand-independent mechanisms. Mol Endocrinol. 2008; 22:1812-24.
Hong J, Stubbins R E, Smith R R, Harvey A E, Nunez N P. Differential susceptibility to obesity between male, female and ovariectomized female mice. Nutr J. 2009; 8:11.
Irahara N, Miyoshi Y, Taguchi T, Tamaki Y, Noguchi S. Quantitative analysis of aromatase mRNA expression derived from various promoters (I.4, I.3, PII and I.7) and its association with expression of TNF-alpha, IL-6 and COX-2 mRNAs in human breast cancer. Int J. Cancer. 2006; 118:1915-21.
Karuppu D, Kalus A, Simpson E R, Clyne C. Aromatase and prostaglandin inter-relationships in breast adipose tissue: significance for breast cancer development. Breast Cancer Res Treat. 2002; 76:103-9.
Kern P A, Ranganathan S, Li C, Wood L, Ranganathan G. Adipose tissue tumor necrosis factor and interleukin-6 expression in human obesity and insulin resistance. Am J Physiol Endocrinol Metab. 2001; 280:E745-51.
Key T J, Appleby P N, Reeves G K, Roddam A, Dorgan J F, Longcope C, et al. Body mass index, serum sex hormones, and breast cancer risk in postmenopausal women. J Natl Cancer Inst. 2003; 95:1218-26.
Kulkarni S, Rader J S, Zhang F, Liapis H, Koki A T, Masferrer J L, et al. Cyclooxygenase-2 is overexpressed in human cervical cancer. Clin Cancer Res. 2001; 7:429-34.
Lorincz A M, Sukumar S. Molecular links between obesity and breast cancer. Endocr Relat Cancer. 2006; 13:279-92.
Lowry O H, Rosebrough N J, Fan A L, Randall R J. Protein measurement with the Folin phenol reagent. J Biol Chem. 1951; 193:265-75.
Majed B, Moreau T, Senouci K, Salmon R J, Fourquet A, Asselain B. Is obesity an independent prognosis factor in woman breast cancer? Breast Cancer Res Treat. 2008; 111:329-42.
McTiernan A, Irwin M, Vongruenigen V. Weight, physical activity, diet, and prognosis in breast and gynecologic cancers. J Clin Oncol. 2010; 28:4074-80.
Murano I, Barbatelli G, Parisani V, Latini C, Muzzonigro G, Castellucci M, et al. Dead adipocytes, detected as crown-like structures, are prevalent in visceral fat depots of genetically obese mice. J Lipid Res. 2008; 49:1562-8.
Olefsky J M, Glass C K. Macrophages, inflammation, and insulin resistance Annu Rev Physiol. 2010; 72:219-46.
Petrelli J M, Calle E E, Rodriguez C, Thun M J. Body mass index, height, and postmenopausal breast cancer mortality in a prospective cohort of US women. Cancer Causes Control. 2002; 13:325-32.
Protani M, Coory M, Martin J H. Effect of obesity on survival of women with breast cancer: systematic review and meta-analysis. Breast Cancer Res Treat. 2010; 123:627-35.
Purohit A, Newman S P, Reed M J. The role of cytokines in regulating estrogen synthesis: implications for the etiology of breast cancer. Breast Cancer Res. 2002; 4:65-9.
Rodbell M. Metabolism of Isolated Fat Cells. I. Effects of Hormones on Glucose Metabolism and Lipolysis. J Biol Chem. 1964; 239:375-80.
Rose D P, Vona-Davis L. Influence of obesity on breast cancer receptor status and prognosis. Expert Rev Anticancer Ther. 2009; 9:1091-101.
Salama S A, Kamel M W, Diaz-Arrastia C R, Xu X, Veenstra T D, Salih S, et al. Effect of tumor necrosis factor-alpha on estrogen metabolism and endometrial cells: potential physiological and pathological relevance. J Clin Endocrinol Metab. 2009; 94:285-93.
Santen R J, Brodie H, Simpson E R, Siiteri P K, Brodie A. History of aromatase: saga of an important biological mediator and therapeutic target. Endocr Rev. 2009; 30:343-75.
Sestak I, Distler W, Forbes J F, Dowsett M, Howell A, Cuzick J. Effect of body mass index on recurrences in tamoxifen and anastrozole treated women: an exploratory analysis from the ATAC trial. J Clin Oncol. 2010; 28:3411-5.
Sinicrope F A, Dannenberg A J. Obesity and breast cancer prognosis: weight of the evidence. J Clin Oncol. 2011; 29:4-7.
Subbaramaiah K, Howe L K, Bhardwaj P, Du B, Gravaghi C, Yantiss R K, et al. Obesity is associated with inflammation and elevated aromatase expression in the mouse mammary gland Cancer Prey Res (Phila Pa.). 2011; 4:329-346
Subbaramaiah K, Howe L R, Port E R, Brogi E, Fishman J, Liu C H, et al. HER-2/neu status is a determinant of mammary aromatase activity in vivo: evidence for a cyclooxygenase-2-dependent mechanism. Cancer Res. 2006; 66:5504-11.
Subbaramaiah K, Hudis C, Chang S H, Hla T, Dannenberg A J. EP2 and EP4 receptors regulate aromatase expression in human adipocytes and breast cancer cells. Evidence of a BRCA1 and p300 exchange. J Biol Chem. 2008; 283:3433-44.
Suganami T, Ogawa Y. Adipose tissue macrophages: their role in adipose tissue remodeling. J Leukoc Biol. 2010; 88:33-9.
van Kruijsdijk R C, van der Wall E, Visseren F L. Obesity and cancer: the role of dysfunctional adipose tissue. Cancer Epidemiol Biomarkers Prey. 2009; 18:2569-78.
Vozarova B, Weyer C, Hanson K, Tataranni P A, Bogardus C, Pratley R E. Circulating interleukin-6 in relation to adiposity, insulin action, and insulin secretion. Obes Res. 2001; 9:414-7.
Wake D J, Strand M, Rask E, Westerbacka J, Livingstone D E, Soderberg S, et al. Intra-adipose sex steroid metabolism and body fat distribution in idiopathic human obesity. Clin Endocrinol (Oxf). 2007; 66:440-6.
Weisberg S P, McCann D, Desai M, Rosenbaum M, Leibel R L, Ferrante A W, Jr. Obesity is associated with macrophage accumulation in adipose tissue. J Clin Invest. 2003; 112:1796-808.
Whiteman M K, Hillis S D, Curtis K M, McDonald J A, Wingo P A, Marchbanks P A. Body mass and mortality after breast cancer diagnosis. Cancer Epidemiol Biomarkers Prey. 2005; 14:2009-14.
Xu H, Barnes G T, Yang Q, Tan G, Yang D, Chou C J, et al. Chronic inflammation in fat plays a crucial role in the development of obesity-related insulin resistance. J Clin Invest. 2003; 112:1821-30.
Yang P, Chan D, Felix E, Madden T, Klein R D, Shureiqi I, et al. Determination of endogenous tissue inflammation profiles by LC/MS/MS: COX- and LOX-derived bioactive lipids. Prostaglandins Leukot Essent Fatty Acids. 2006; 75:385-95.
Zhao Y, Agarwal V R, Mendelson C R, Simpson E R. Estrogen biosynthesis proximal to a breast tumor is stimulated by PGE2 via cyclic AMP, leading to activation of promoter II of the CYP19 (aromatase) gene. Endocrinology. 1996; 137:5739-42.
Zhao Y, Agarwal V R, Mendelson C R, Simpson E R. Transcriptional regulation of CYP19 gene (aromatase) expression in adipose stromal cells in primary culture. J Steroid Biochem Mol Biol. 1997; 61:203-10.
Zuckennann et al., 1994; Med. Chem. 37: 2678-85.

1. A method for determining cancer risk or prognosis in a patient which comprises detecting the presence of crown-like structures (CLS) in adipose tissue in a tissue sample from said patient, said adipose tissue being in or around an epithelial-derived tissue or organ or said adipose tissue being associated with a lymph node metastasis arising from an epithelial-derived cancer, wherein the presence of CLS indicates poor prognosis or an increased cancer risk.

2. The method of claim 1 wherein detecting CLS comprises obtaining said tissue sample, preparing one or more tissue sections from said sample, staining said one or more sections histochemically or with a macrophage-specific marker and identifying CLS in said one or more sections.

3. The method of claim 2, wherein the number of CLS in said one or more tissue sections are quantitated.

4. The method of claim 1, wherein said cancer is breast cancer, head and neck carcinoma, prostate cancer, colorectal cancer, kidney cancer, pancreatic cancer or liver cancer.

5-8. (canceled)

9. The method of claim 1 which comprises quantifying the number of CLS in a sample of adipose tissue from the patient, wherein a patient with a CLS index of 0.2 to 1.0 has a worse prognosis or is at greater risk for cancer than a patient with a CLS index less than 0.2.

10. (canceled)

11. The method of claim 9 wherein detecting CLS comprises obtaining said tissue sample, preparing one or more tissue sections from said sample, staining said one or more sections histochemically or with a macrophage-specific marker and identifying CLS in said one or more sections.

12. The method of claim 9, wherein said cancer is breast cancer, head and neck carcinoma, prostate cancer, colorectal cancer, kidney cancer, pancreatic cancer or liver cancer.

13-16. (canceled)

17. A method for treating cancer in a patient which comprises
(a) determining whether CLS are present in adipose tissue of a patient by detecting the presence of CLS in said adipose tissue, said adipose tissue being obtained from in or around an epithelial-derived tissue or organ or said adipose tissue being associated with a lymph node metastasis arising from an epithelial-derived cancer, and when CLS are detected,
(b) administering to said patient a therapeutically-effective dose of a therapeutic agent that reduces the number or function of CLS or the biological consequences of CLS, or any combination thereof.

18. The method of claim 17, wherein said therapeutic agent is selected from the group consisting of a Toll-like receptor antagonist, an NF-kB inhibitor, a CDK5 inhibitor, a TNF-α inhibitor, a COX-2 inhibitor, an IL-1 inhibitor, an aromatase inhibitor, a PI3K inhibitor, a Rho-kinase inhibitor, an Akt inhibitor, an mTOR inhibitor, an IGF-1R antagonist, an anti-leptin compound, an AMPK activator, a statin, and resveratrol or other polyphenols.

19. The method of claim 17, wherein detecting CLS comprises obtaining a tissue sample, preparing one or more tissue sections from said sample, staining said one or more sections histochemically or with a macrophage-specific marker and identifying CLS in said one or more sections.

20. The method of claim 19, wherein the number of CLS in said one or more tissue sections are quantitated.

21. The method of claim 17, wherein said cancer is breast cancer, head and neck carcinoma, prostate cancer, colorectal cancer, kidney cancer, pancreatic cancer or liver cancer.

22-27. (canceled)

28. The method of claim 1, wherein the method is for determining prognosis of a patient with breast cancer and comprises detecting CLS in breast adipose tissue, wherein presence of CLS indicates a worse prognosis.

29. The method of claim 28, wherein detecting CLS comprises obtaining a tissue sample, preparing one or more tissue sections from said sample, staining said one or more sections histochemically or with a macrophage-specific marker and identifying CLS in said one or more sections.

30. The method of claim 29, wherein the amount of CLS in said one or more tissue sections are quantitated.

31. The method of claim 30, wherein said CLS are quantitated by calculating a CLS-index for said patient, wherein individuals with high numbers of CLS or a higher CLS-index have a worse prognosis or are at greater risk for developing hormone receptor-positive breast cancer or worse prognosis than individuals with low numbers of CLS, no CLS or a lower CLS-index.

32. (canceled)

33. The method of claim 28, which further comprises determining a patient's body mass index (BMI), wherein presence of CLS together with higher patient BMI indicates increased cancer risk.

34. An in vitro method to screen a compound for anti-cancer activity which comprises
(a) culturing macrophages with said compound and a saturated fatty acid in an amount and for a time sufficient to produce conditioned medium comprising pro-inflammatory mediators;
(b) harvesting said conditioned medium;
(c) culturing cells selected from the group consisting of preadipocytes, adipocytes, fibroblasts, epithelial cells and tumor cells in the presence of said conditioned medium for a time to induce a cancer-related response to said conditioned medium; and
(d) detecting said response, wherein inhibition or reduction of said response, relative to similarly treated control cultures without said compound, indicates said compound has anti-cancer activity.

35. The method of claim 34, wherein said response is inducing aromatase activity, and inhibition or reduction of aromatase activity is detected.

36. The method of claim 35, wherein said cells are preadipocytes or tumor cells.

37. The method of claim 34 wherein said response is an increase in cell proliferation, an increase in cell motility, an increase in invasiveness, an increase in NFκB activity, or a decrease in apoptosis.

38-43. (canceled)

44. The method of claim 34, wherein said saturated fatty is selected from the group consisting of lauric acid, myristic acid, palmitic acid and stearic acid.

45. The method of claim 35 wherein aromatase activity can be determined from the level of aromatase expression in said cells or by measuring the enzymatic activity of aromatase produced by said cells.

46. A method for determining a compound's activity against CLS which comprises (a) screening said compound in a dietary or genetic model of obesity in mice; and (b) determining whether said compound inhibits or reduces the number or formation of CLS in breast adipose tissue of said mice or a functional consequence of CLS in said mice.

47. The method of claim 46, which comprises injecting said mice with tumor cells and determining whether tumor formation or growth is inhibited relative to untreated mice, wherein a compound which inhibits or reduces CLS formation or the functional consequences of CLS-B is a candidate agent for reducing cancer progression.

48. A method of screening a compound for a medical benefit which comprises
(a) treating lean mice and obese mice with said compound in an amount and for a time sufficient to assess the effects of said compound; and
(b) determining whether said compound preferentially inhibits or reduces formation of CLS-B or functional consequences of CLS-B in the obese mice, wherein a compound which inhibits or reduces formation of CLS-B or functional consequences of CLS-B provides a medical benefit.

49. The method of claim 48, which comprises injecting said mice with tumor cells and determining whether tumor formation or growth is inhibited relative to untreated mice, wherein a compound which inhibits or reduces CLS formation or the functional consequences of CLS-B is a candidate agent for reducing cancer progression.

50. The method of claim 48, wherein said lean mice are ovary intact mice fed a low fat diet.

51. The method of claim 48, wherein said obese mice are ovariectomized mice fed a high fat diet.

52. The method of claim 48, wherein said medical benefit is anti-cancer activity, anti-diabetogenic activity or anti-obesity activity.

53. (canceled)

54. The method of claim 1, wherein the method is for determining prognosis of a patient with a head and neck carcinoma by detecting CLS in adipose tissue found with lymph node metastasis associated with said carcinoma, wherein presence of CLS indicates a worse prognosis or an increased risk of metastasis.

55. (canceled)


SEQUENCE LISTING (30 sequences)

SEQ ID NO: 1 - DNA, 20 bp, Artificial Sequence
  Synthetic Oligonucleotide primer
attctttgcccagcacttca

SEQ ID NO: 2 - DNA, 20 bp, Artificial Sequence
  Synthetic Oligonucleotide primer
gggatacacctctccaccaa

SEQ ID NO: 3 - DNA, 21 bp, Artificial Sequence
  Synthetic Oligonucleotide primer
ccagaccctcacactcagatc

SEQ ID NO: 4 - DNA, 21 bp, Artificial Sequence
  Synthetic Oligonucleotide primer
cacttggtggtttgctacgac

SEQ ID NO: 5 - DNA, 20 bp, Artificial Sequence
  Synthetic Oligonucleotide primer
tgggcctcaaaggaaagaat

SEQ ID NO: 6 - DNA, 20 bp, Artificial Sequence
  Synthetic Oligonucleotide primer
caggcttgtgctctgcttgt

SEQ ID NO: 7 - DNA, 20 bp, Artificial Sequence
  Synthetic Oligonucleotide primer
aagctctgacgggccctggt

SEQ ID NO: 8 - DNA, 20 bp, Artificial Sequence
  Synthetic Oligonucleotide primer
acgtagcccgaggtgtcggt

SEQ ID NO: 9 - DNA, 19 bp, Artificial Sequence
  Synthetic Oligonucleotide primer
ggtgggccttcctaacgag

SEQ ID NO: 10 - DNA, 21 bp, Artificial Sequence
  Synthetic Oligonucleotide primer
gaccacatcaggctcaatgct

SEQ ID NO: 11 - DNA, 20 bp, Artificial Sequence
  Synthetic Oligonucleotide primer
ctgcccaggagagaaatgag

SEQ ID NO: 12 - DNA, 20 bp, Artificial Sequence
  Synthetic Oligonucleotide primer
cagggtatgagggttctcca

SEQ ID NO: 13 - DNA, 20 bp, Artificial Sequence
  Synthetic Oligonucleotide primer
aatgtgtccgtcgtggatct

SEQ ID NO: 14 - DNA, 20 bp, Artificial Sequence
  Synthetic Oligonucleotide primer
catcgaaggtggaagagtgg

SEQ ID NO: 15 - DNA, 20 bp, Artificial Sequence
  Synthetic Oligonucleotide primer
cccttgggtgtcaaaggtaa

SEQ ID NO: 16 - DNA, 20 bp, Artificial Sequence
  Synthetic Oligonucleotide primer
gccctcgcttatgatctgtc

SEQ ID NO: 17 - DNA, 20 bp, Artificial Sequence
  Synthetic Oligonucleotide primer
ctgctgcactttggagtgat

SEQ ID NO: 18 - DNA, 20 bp, Artificial Sequence
  Synthetic Oligonucleotide primer
agatgatctgactgcctggg

SEQ ID NO: 19 - DNA, 20 bp, Artificial Sequence
  Synthetic Oligonucleotide primer
ggacaagctgaggaagatgc

SEQ ID NO: 20 - DNA, 20 bp, Artificial Sequence
  Synthetic Oligonucleotide primer
tcgttatcccatgtgtcgaa

SEQ ID NO: 21 - DNA, 21 bp, Artificial Sequence
  Synthetic Oligonucleotide primer
cacatcctcaataccaggtcc

SEQ ID NO: 22 - DNA, 20 bp, Artificial Sequence
  Synthetic Oligonucleotide primer
cagagatccagactcgcatg

SEQ ID NO: 23 - DNA, 20 bp, Artificial Sequence
  Synthetic Oligonucleotide primer
agaaaatctggcaccacacc

SEQ ID NO: 24 - DNA, 20 bp, Artificial Sequence
  Synthetic Oligonucleotide primer
agaggcgtacagggatagca

SEQ ID NO: 25 - DNA, 21 bp, Artificial Sequence
  Synthetic Oligonucleotide primer
gatccggaggagattccttga

SEQ ID NO: 26 - DNA, 34 bp, Artificial Sequence
  Synthetic Oligonucleotide primer
acactccaggcacttaagggtcccgactcaagta

SEQ ID NO: 27 - DNA, 25 bp, Artificial Sequence
  Synthetic Oligonucleotide primer
cgtgggaaaatccagtattttaatg

SEQ ID NO: 28 - DNA, 25 bp, Artificial Sequence
  Synthetic Oligonucleotide primer
caaatgtatcaccatgcaaatatgc

SEQ ID NO: 29 - DNA, 25 bp, Artificial Sequence
  Synthetic Oligonucleotide primer
aaagctatgtatgtatgtgctgcat

SEQ ID NO: 30 - DNA, 24 bp, Artificial Sequence
  Synthetic Oligonucleotide primer
aaccgagagaaccttcctttttat